(19) United States
(12) Patent Application Publication
(10) Pub. No.: US 2011/0104771 A1
(43) Pub. Date: May 5, 2011

(54) PROCESS FOR THE PRODUCTION OF A DICARBOXYLIC ACID

(76) Inventors: Rene Verwall, Nootdorp (NL); Liang Wu, Delft (NL); Robbertus Antonius Damveld, Berkel en Rodenrijs (NL); Cornells Maria Jacobus Sagt, Utrecht (NL)

(21) Appl. No.: 12/867,818

(22) PCT Filed: Feb. 13, 2009
(86) PCT No.: PCT/EP2009/051717

§ 371 Date: Nov. 1, 2010

(30) Foreign Application Priority Data
Feb. 15, 2008 (EP) 08101656.0

(51) Int. Cl. C12P 7/46; C12N 1/19

(52) U.S. Cl. 435/145; 435/254.21

(57) ABSTRACT

The present invention relates to a process for the production of a dicarboxylic acid wherein a eukaryotic cell is fermented in a suitable fermentation medium. The invention further relates to a eukaryotic cell comprising a nucleotide sequence encoding an enzyme which catalyses the conversion of isocitric acid to succinic acid, and a nucleotide sequence encoding an enzyme which catalyses the conversion of glyoxylic acid to malic acid.

The present invention relates to a process for the production of a dicarboxylic acid and a eukaryotic cell comprising an enzyme that catalyses the conversion of isocitric acid to succinic acid and an enzyme that catalyses the conversion of glyoxylic acid to malic acid.

The 4-carbon dicarboxylic acids, malic acid, fumaric acid and succinic acid, are potential precursors for numerous chemicals and have numerous applications in pharmaceutical, cosmetic, food, feed or chemical industry.

Until date, malic acid, fumaric acid and succinic acid are predominantly produced through (petro) chemical processes, which are considered harmful to the environment and costly.

The fermentative production of dicarboxylic acids such as malic acid, fumaric acid and succinic acid may be an attractive alternative process for the production of these dicarboxylic acids, wherein renewable feedstock as a carbon source may be used.

A number of different bacteria such as Escherichia coli, and the rumen bacteria Actinobacillus, Anaerobiospirillum, Bacteroides, Mannheimia, or Succinimonas, sp. are known to produce succinic acid. Metabolic engineering of these bacterial strains have improved the succinic acid yield and/or productivity, or reduced the by-product formation.

WO2007/061590 discloses a pyruvate decarboxylase negative yeast for the production of malic acid and/or succinic acid which is transformed with a pyruvate carboxylase enzyme or a phosphoenolpyruvate carboxylase, a malate dehydrogenase enzyme, and a malic acid transporter protein.

Despite the improvements that have been made in the fermentative production of dicarboxylic acids, there remains a need for an improved production process of dicarboxylic acids.

The aim of the present invention is an improved process for the production of a dicarboxylic acid.

The aim is achieved according to the invention by a process for the production of a dicarboxylic acid comprising fermenting a eukaryotic cell in a suitable fermentation medium, wherein the eukaryotic cell comprises an enzyme which catalyses the

conversion of isocitric acid to succinic acid, and producing the dicarboxylic acid wherein succinic acid is produced in the cytosol. As understood herein, the conversion of isocitric acid to succinic acid comprises the formation of glyoxylic acid. Preferably, the eukaryotic cell in the process of the invention comprises an enzyme that catalyses the conversion of isocitric acid to succinic acid and glyoxylic acid.

Surprisingly, an increased amount of dicarboxylic acid was produced in the process according to the present invention, compared to a process wherein a eukaryotic cell is fermented which does not comprise an enzyme which catalyses the conversion of isocitric acid to succinic acid, wherein succinic acid is produced in the cytosol.

A suitable dicarboxylic acid that may be produced in the process according to the present invention is a 4-carbon dicarboxylic acid selected from malic acid, fumaric acid, and succinic acid. Preferably, the dicarboxylic acid is fumaric acid or succinic acid, in particular succinic acid.

As used herein, the terms dicarboxylic acid, malic acid, fumaric acid, succinic acid, isocitric acid and glyoxylic acid also cover the compounds dicarboxylate, malate, fumarate, succinate, isocitrate and glyoxylate, i.e. the ionic form of the acids, and salts, esters, or ethers thereof and the terms may be used interchangeably. The acid form is the hydrogenated form of the ionic form, and is influenced by the pH.

The eukaryotic cell fermented in the process according to the present invention may be a wild-type or a recombinant eukaryotic cell. As used herein, a recombinant eukaryotic cell is defined as a cell which contains a nucleotide sequence and/or protein, or is transformed or genetically modified with a nucleotide sequence that does not naturally occur in the yeast, or it contains additional copy or copies of an endogenous nucleic acid sequence (or protein). Commonly, a eukaryotic cell of the invention is a recombinant eukaryotic cell.

The enzyme which catalyses the conversion of isocitrate to succinate, may be any suitable heterologous or homologous enzyme. Preferably, the enzyme is an isocitrate lyase (EC 4.1.3.1).

The term “homologous” as used herein, refers to a nucleic acid (DNA or RNA) or polypeptide that is endogenous to, or found in nature in a cell or organism, genome, DNA, or RNA sequence.

The term “heterologous” as used herein, refers to a nucleic acid or polypeptide that is exogenous to, or does not occur naturally as part of the organism, cell, genome DNA or RNA sequence in which it is present.

For the production of a dicarboxylic acid such as succinic acid or malic acid in the cytosol, cytosolic localisation of the enzyme that catalyses the production of a dicarboxylic acid may be needed. The enzyme may be naturally present in the cytosol or cytosolic localisation may be obtained by deletion of a targeting sequence, for example a peroxisomal (or mitochondrial) targeting signal from the enzyme, in order to obtain cytosolic activity of the enzyme. The presence of a targeting signal may be identified by known methods in the art, for instance as determined by the method disclosed by Schlüter et al, Nucleic acid Research 2007, Vol 25, D815-D822. A peroxisomal targeting signal is a region in a peroxisomal protein that binds to a receptor, which receptor directs the protein to the peroxisome. Peroxisomal proteins are synthesised in the cytosol. Deletion of a peroxisimal targeting signal will prevent peroxisomal targeting.

Preferably, a gene expressed in a eukaryotic cell of the invention encoding an enzyme that catalyses the production of a dicarboxylic acid, eg. an enzyme that catalyses the conversion of isocitrate to succinate or glyoxylate to malate, is expressed in the presence of a fermentable sugar.

Expression of a gene in the presence of a fermentable sugar may occur naturally, or may be obtained by deletion of glucose repression of the enzyme, for instance by replacing a promoter sensitive to glucose repression with a non-glucose repression sensitive promoter. Such promoters are known to the skilled person in the art.

Glucose repression is the repression of certain sugar-metabolizing operons in favour of glucose utilization wherein glucose is the predominant carbon source in the environment of the cell.

Preferably, an enzyme that catalyses the production of a dicarboxylic acid, eg. an enzyme that catalyses the conversion of isocitrate to succinate in the cytosol, is an enzyme that is not degraded or suppressed in the presence of a fermentable sugar, i.e. an enzyme that is not subjected to catabolite inactivation.

A nucleotide sequence encoding an enzyme that catalyses the conversion of isocitrate to succinate, wherein the enzyme is active in the cytosol, may encode a homologous or heterologous enzyme. Preferably, the enzyme is a heterologous enzyme.

Preferably, a eukaryotic cell in the process of the invention comprises an enzyme catalysing the conversion of isocitrate to succinate (and glyoxylate), which has at least 30%, preferably at least 40, 50, 60, 70, 75, 80, 85, 90, 95, 96, 97, 98, 99 or 100% sequence identity with the amino acid of SEQ ID NO: 1. Preferably, the enzyme catalysing the conversion of isocitrate to succinate is active in the cytosol.

The process according to the present invention was found particularly advantageous when the eukaryotic cell was fermented in a fermentation medium comprising a fermentable sugar. Fermentable sugars may be glucose, fructose, sucrose, maltose, galactose, raffinose, arabinose, xylose, or xylulose.

During the course of the process for the production of a dicarboxylic acid of the invention, a carbon source is converted to a dicarboxylic acid in a eukaryotic cell and secreted by the cell into the medium.

In another aspect the present invention relates to a process for the production of a dicarboxylic acid according to the present invention, wherein a eukaryotic cell is fermented which comprises a nucleotide sequence encoding an enzyme, which catalyses the conversion of glyoxylic acid to malic acid, wherein malic acid is produced in the cytosol.

Preferably, the process for the production of a dicarboxylic acid according to the present invention is a process wherein a eukaryotic cell is fermented which comprises an enzyme which catalyses the conversion of isocitric acid to succinic acid (and glyoxylic acid) and a second enzyme which catalyses the conversion of glyoxylic acid to malic acid, wherein succinic acid and malic acid are produced in the cytosol.

Surprisingly, it was found in the process according to the present invention that when succinic acid and/or malic acid were/was produced in the cytosol an increased amount of a dicarboxylic acid, in particular succinic acid was produced by the eukaryotic cell.

Preferably, the enzyme that catalyses the conversion of glyoxylate to malate in the eukaryotic cell of the invention is a malate synthase (EC 2.3.3.9). Cytosolic activity of an enzyme catalysing the conversion of glyoxylate to malate may be obtained as described herein above, preferably, by deletion of a peroxisomal targeting signal. In the event the malate synthase is a Saccharomyces cerevisiae malate synthase, preferably the native malate synthase is altered by the deletion of the SKL carboxy-terminal sequence.

Preferably, the nucleotide sequence encoding an enzyme that catalyses the conversion of glyoxylate to malate in the cytosol has at least 40%, preferably at least 50, 60, 70, 75, 80, 85, 90, 95, 96, 97, 98, 99, or 100% sequence identity to SEQ ID NO: 5.

Sequence identity is herein defined as a relationship between two or more amino acid (polypeptide or protein) sequences or two or more nucleic acid (polynucleotide) sequences, as determined by comparing the sequences. Usually, sequence identities are compared over the whole length of the sequences compared. In the art, “identity” also means the degree of sequence relatedness between amino acid or nucleic acid sequences, as the case may be, as determined by the match between strings of such sequences.

Preferred methods to determine identity are designed to give the largest match between the sequences tested. Methods to determine identity are codified in publicly available computer programs. Preferred computer program methods to determine identity and similarity between two sequences include e.g. the BLASTP, BLASTN), publicly available from NCBI and other sources (BLAST Manual, Altschul, S., et al., NCBI NLM NIH Bethesda, Md. 20894). Preferred parameters for amino acid sequences comparison using BLASTP are gap open 11.0, gap extension 1, Blosum 62 matrix.

A nucleotide sequence encoding an enzyme expressed in the cell of the invention may also be defined by their capability to hybridise with the nucleotide sequences encoding an enzyme of SEQ ID NO.'s: 1 or 5, under moderate, or preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC (sodium chloride, sodium citrate) or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

Moderate conditions are herein defined as conditions that allow a nucleic acid sequence of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

To increase the likelihood that an enzyme is expressed in active form in a eukaryotic cell of the invention, the corresponding encoding nucleotide sequence may be adapted to optimise its codon usage to that of the chosen eukaryotic host cell. Several methods for codon optimisation are known in the art. A preferred method to optimise codon usage of the nucleotide sequences to the eukaryotic cell according to the present invention is codon pair optimization technology as disclosed in WO2008/000632.

A eukaryotic cell in the process for the production of a dicarboxylic acid may be any suitable yeast or filamentous fungus. Preferably, a eukaryotic cell in the process according to the present invention belongs to the genera selected from the group consisting of Saccharomyces, Aspergillus, Penicillium, Pichia, Kluyveromyces, Candida, Hansenula, Trichosporon, Trichoderma, Rhizopus, and Zygosaccharomyces. Preferably, the eukaryotic cell belongs to a species Saccharomyces cerevisiae, Saccharomyces uvarum, Saccharomyces bayanus, Aspergillus niger, Penicillium chrysogenum, P. symplissicum, Pichia stipidis, P. pastoris, Kluyveromyces marxianus, K. lactis, K. thermotolerans, Trichoderma reesii, Candida sonorensis, C. glabrata, Rhizopus oryzae and Zygosaccharomyces bailii. The eukaryotic cell according to the present invention preferably belongs to a Saccharomyces sp., preferably a Saccharomyces cerevisiae.

The process for the production of a dicarboxylic acid according to the present invention may be run under aerobic, anaerobic, micro-aerophilic or oxygen limited conditions or a combination of aerobic and anaerobic/micro-aerophilic conditions. It may be preferred to grow the eukaryotic cell under aerobic conditions to a certain cell density and subsequently produce a dicarboxylic acid under anaerobic conditions, or micro-aerophilic or oxygen limited conditions.

An anaerobic fermentation process is herein defined as a fermentation process run in the absence of oxygen or in which substantially no oxygen is consumed, preferably less than 5, 2.5 or 1 mmol/L/Jh.

An oxygen-limited fermentation process is a process in which the oxygen consumption is limited by the oxygen transfer from the gas to the liquid. The degree of oxygen limitation is determined by the amount and composition of the ingoing gasflow as well as the actual mixing/mass transfer properties of the fermentation equipment used. Preferably, in a process under oxygen-limited conditions, the rate of oxygen consumption is at least 5.5, more preferably at least 6 and even more preferably at least 7 mmol/Uh.

The process for the production of a dicarboxylic acid according to the present invention may be carried out at any suitable pH between 1 and 9. Preferably, the pH in the fermentation broth is between 2 and 7, preferably between 2.5 and 6, preferably between 3 and 5.5, preferably between 3.5 and 5. It was found advantageous to be able to carry out the process according to the present invention at low pH, since this prevents bacterial contamination and less alkaline salts are needed for titration to maintain the pH at a desired level in the process for the production of succinate.

A suitable temperature at which the process according to the present invention may be carried out is between 5 and 60° C., preferably between 10 and 50° C., more preferably between 15 and 35° C., more preferably between 18° C. and 30° C. The skilled person in the art knows the optimal temperatures for fermenting a specific eukaryotic cell.

The process for the production of a dicarboxylic acid according to the present invention may be carried out in any suitable volume, preferably on an industrial scale. Preferably the process of the invention is carried out in a volume of at least 10 ml, 100 ml, 1 l, 10 l, 100 l, preferably at least 1 m³ (cubic metre), 10 m³ (cubic metre) or 100 m³ (cubic metre), and usually below 1000 m³ (cubic metre).

The fermentation medium may comprise any suitable component which allows optimal growth of and production of a dicarboxylic acid by a eukaryotic cell in the process according to the present invention, which are know to the skilled person. Preferably the fermentation medium comprises a source of carbon dioxide, for instance in the form of calcium carbonate or by flowing gaseous carbon dioxide through the medium.

In a preferred embodiment the process for the production of a dicarboxylic acid according to the present invention comprises recovering the dicarboxylic acid produced from the fermentation medium. Recovery of a dicarboxylic acid, such as malic acid, fumaric acid or succinic acid, from the fermentation medium may be carried out by any suitable method known in the art, for instance by crystallisation, ammomium precipitation or ion exchange technology. Preferably, the process for the production of a dicarboxylic acid further comprises purifying the dicarboxylic acid.

In another preferred embodiment, the process of the present invention comprises using a (fermentatively) produced dicarboxylic acid for the preparation of a product comprising a dicarboxylic acid or a derivative thereof. A derivative may for instance be esters, ethers, aldehydes, or salts of a dicarboxylic acid. Suitable products may for instance be a pharmaceutical, cosmetic, food, feed, or chemical product. Succinic acid and fumaric acid may be converted into their corresponding polyester polymers, such as polybutylenesuccinate (PBS). Succinic acid may also be converted by hydrogenation to 1,4-butanediol.

In another aspect, the present invention relates to a eukaryotic cell comprising a nucleotide sequence encoding a first enzyme which catalyses the conversion of isocitric acid to succinic acid, and a nucleotide sequence encoding a second enzyme which catalyses the conversion of glyoxylic acid to malic acid, wherein the first enzyme and the second enzyme are active in the cytosol. Commonly, the first enzyme in the eukaryotic cell of the invention catalyses the conversion of isocitric acid to succinic acid and glyoxylic acid.

Surprisingly, it was found that the eukaryotic cell according to the present invention produces an increased amount of a dicarboxylic acid, as compared to a eukaryotic cell which does not comprise an enzyme which catalyses the conversion of isocitric acid to succinic acid, and an enzyme which catalyses the conversion of glyoxylic acid to malic acid wherein both enzymes are active in the cytosol. A eukaryotic cell of the invention may advantageously be used in a process of the invention. The eukaryotic cell according to the present invention may be a yeast or a filamentous fungus, preferably according to a genus and species as defined herein above.

Preferred embodiments of an enzyme which catalyses the conversion of isocitrate to succinate and of an enzyme which catalyses the conversion of glyoxylate to malate in the eukaryotic cell according to the present invention and other preferred embodiments are as defined herein above.

Preferably, the eukaryotic cell according to the present invention is a Saccharomyces cerevisiae, preferably a Saccharomyces cerevisiae comprising a nucleotide sequence of SEQ ID NO: 6 encoding an enzyme having isocitrate lyase activity and a nucleotide sequence of SEQ ID NO: 7 encoding an enzyme having malate synthase activity.

Usually, a nucleotide sequence encoding an enzyme is operably linked to a promoter that causes sufficient expression of the corresponding nucleotide sequence in the eukaryotic cell according to the present invention to confer to the cell the ability to produce a dicarboxylic acid.

As used herein, the term “operably linked” refers to a linkage of polynucleotide elements (or coding sequences or nucleic acid sequence) in a functional relationship. A nucleic acid sequence is “operably linked” when it is placed into a functional relationship with another nucleic acid sequence. For instance, a promoter or enhancer is operably linked to a coding sequence if it affects the transcription of the coding sequence.

The term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences known to one of skilled in the art. A “constitutive” promoter is a promoter that is active under most environmental and developmental conditions. An “inducible” promoter is a promoter that is active under environmental or developmental regulation.

The promoter that could be used to achieve expression of a nucleotide sequence coding an enzyme in a eukaryotic cell of the invention, may be not native to the nucleotide sequence coding for the enzyme to be expressed, i.e. a promoter that is heterologous to the nucleotide sequence (coding sequence) to which it is operably linked. Preferably, the promoter is homologous, i.e. endogenous to the host cell.

Suitable promoters in eukaryotic cells are known to the skilled man in the art. Suitable promoters may be, but are not limited to TDH, GPDA, GAL7, GAL10, or GAL1, CYC1, HIS3, ADH1, PGL, PH05, ADC1, TRP1, URA3, LEU2, ENO, TPI, AOX1, PDC, GPD1, PGK1, and TEF1.

Usually a nucleotide sequence encoding an enzyme comprises a terminator. Any terminator, which is functional in the cell, may be used in the present invention. Preferred terminators are obtained from natural genes of the host cell. Suitable terminator sequences are well known in the art. Preferably, such terminators are combined with mutations that prevent nonsense mediated mRNA decay in the host cell of the invention (see for example: Shirley et al., 2002, Genetics 161:1465-1482).

The nucleotide sequence encoding an enzyme that catalyses the conversion of isocitrate to succinate and/or glyoxylate to malate may be overexpressed in the eukaryotic cell according to the present invention. There are known methods in the art for overexpression of nucleotide sequences encoding enzymes. A nucleotide sequence encoding an enzyme may be overexpressed by increasing the copy number of the gene coding for the enzyme in the cell, e.g. by integrating additional copies of the gene in the cell's genome, by expressing the gene from a centromeric vector, from an episomal multicopy expression vector or by introducing an (episomal) expression vector that comprises multiple copies of one or more gene(s). Preferably, overexpression of a nucleotide sequence encoding an enzyme according to the invention is achieved with a (strong) constitutive promoter.

In the scope of the present invention, the nucleotide sequence encoding an enzyme may be ligated into a nucleic acid construct, for instance a plasmid, such as a low copy plasmid or a high copy plasmid. The eukaryotic cell according to the present invention may comprise a single, or multiple copies of the nucleotide sequence encoding an enzyme, for instance by multiple copies of a nucleotide construct.

A nucleic acid construct may be maintained episomally and thus comprises a sequence for autonomous replication, such as an autosomal replication sequence. If the eukaryotic cell is of fungal origin, a suitable episomal nucleic acid construct may e.g. be based on the yeast 2μ or pKD1 plasmids (Gleer et al., 1991, Biotechnology 9: 968-975), or the AMA plasmids (Fierro et al., 1995, Curr Genet. 29:482-489). Alternatively, each nucleic acid construct may be integrated in one or more copies into the genome of the eukaryotic cell. Integration into the cell's genome may occur at random by non-homologous recombination but preferably, the nucleic acid construct may be integrated into the cell's genome by homologous recombination as is well known in the art.

In a preferred embodiment, a eukaryotic cell according to the present invention further comprises a nucleotide sequence encoding a heterologous PEP carboxykinase (EC 4.1.1.49) catalysing the reaction from phosphoenolpyruvate to oxaloacetate. Preferably, a PEP carboxykinase that is derived from bacteria, more preferably the enzyme having PEP carboxykinase activity is derived from Escherichia coli, Mannheimia sp., Actinobacillus sp., or Anaerobiospirillum sp., more preferably Mannheimia succiniciproducens, Actinobacillus succinogenes, or Anaerobiospirillum succiniciproducens.

In another preferred embodiment a eukaryotic cell according to the present invention further comprises a nucleotide sequence encoding a malate dehydrogenase (MDH) which is active in the cytosol upon expression of the nucleotide sequence. A cytosolic MDH may be any suitable homologous or heterologous malate dehydrogenase. The MDH may be a S. cerevisiae MDH3 or S. cerevisiae MDH1. Preferably, the MDH lacks a peroxisomal or mitochondrial targeting signal in order to localize the enzyme in the cytosol. Alternatively, the MDH is S. cerevisiae MDH2 which has been modified such that it is not inactivated in the presence of glucose and is active in the cytosol. It is known that the transcription of MDH2 is repressed and Mdh2p is degraded upon addition of glucose to glucose-starved cells. Mdh2p deleted for the first 12 amino-terminal amino acids is less-susceptible for glucose-induced degradation (Minard and McAlister-Henn, J. Biol. Chem. 1992 Aug. 25; 267(24):17458-64).

A eukaryotic cell according to the present invention may also comprise a nucleotide sequence encoding an enzyme that catalyses the conversion of malic acid to fumaric acid, which may be a heterologous or homologous enzyme. Preferably, the enzyme is active in the cytosol. An enzyme that catalyses the conversion of malic acid to fumaric acid, for instance a fumarase, may be derived from any suitable origin, preferably from microbial origin, for instance a yeast such as Saccharomyces or a filamentous fungus, such Rhizopus oryzae. Preferably, the nucleotide sequence encoding an enzyme catalyzing the conversion from malic acid to fumaric acid is overexpressed by methods as described herein above.

In another preferred embodiment a eukaryotic cell of the invention expresses a nucleotide sequence encoding an enzyme that catalyses the formation of succinic acid, wherein the nucleotide sequence preferably encodes a NAD(H)-dependent fumarate reductase. Preferably, the NADH-dependent fumarate reductase is a heterologous enzyme, which may be derived from any suitable origin, for instance bacteria, fungi, protozoa or plants. Preferably, the cell according to the invention comprises a heterologous NAD(H)-dependent fumarate reductase, preferably derived from a Trypanosoma sp. for instance a Trypanosoma brucei.

In a preferred embodiment the nucleotide sequence encoding a NAD(H)-dependent fumarate reductase is expressed in the cytosol. Surprisingly, cytosolic activity of the enzyme resulted in an increased productivity of succinic acid by the eukaryotic cell.

It was surprisingly found that additional (over)expression of genes encoding PEP carboxykinase, malate dehydrogenase, NAD(H) fumarate reductase, and/or fumarase as described herein in the eukaryotic cell of the invention resulted in increased succinic acid production levels.

Preferably, a eukaryotic cell according to the present invention is a cell wherein at least one gene encoding alcohol dehydrogenase is not functional. An alcohol dehydrogenase gene that is not functional is used herein to describe a eukaryotic cell, which comprises a reduced alcohol dehydrogenase activity compared to a cell wherein all genes encoding an alcohol dehydrogenase are functional. A gene may become not functional by known methods in the art, for instance by mutation, disruption, or deletion, for instance by the method disclosed by Gueldener et. al. 2002, Nucleic Acids Research, Vol. 30, No. 6, e23. Preferably, the cell is a Saccharomyces cerevisiae, wherein one or more of the genes ADH1 and/or ADH2, encoding alcohol dehydrogenase are inactivated.

Preferably, the cell according to the present invention further comprises at least one gene encoding glycerol-3-phosphate dehydrogenase which is not functional. A glycerol-3-phosphate dehydrogenase gene that is not functional is used herein to describe a eukaryotic cell, which comprises a reduced glycerol-3-phosphate dehydrogenase activity, for instance by mutation, disruption, or deletion of the gene encoding glycerol-3-phosphate dehydrogenase, resulting in a decreased formation of glycerol as compared to a cell wherein the at least one gene encoding glycerol-3-phosphate dehydrogenase is functional. Preferably, the cell is a Saccharomyces cerevisiae, wherein one or more of the genes GPD1 and/or GPD2, encoding glycerol-3-phosphate dehydrogenase are inactivated.

The present invention also relates to a eukaryotic cell transformed such that the cell is capable of producing a dicarboxylic acid by fermenting the cell in a suitable fermentation medium wherein the cell comprises an enzyme catalysing the conversion of isocitric acid to succinic acid (and glyoxylic acid), wherein succinic acid is produced in the cytosol and/or an enzyme that catalyses the conversion of glyoxylic acid to malic acid, wherein malic acid is produced in the cytosol. Preferably, the eukaryotic cell is transformed with enzymes as described above.

Genetic Modifications

Standard genetic techniques, such as overexpression of enzymes in the host cells, genetic modification of host cells, or hybridisation techniques, are known methods in the art, such as described in Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Methods for transformation, genetic modification etc of fungal host cells are known from e.g. EP-A-0 635 574, WO 98/46772, WO 99/60102 and WO 00/37671, WO90/14423, EP-A-0481008, EP-A-0635 574 and U.S. Pat. No. 6,265,186.

The following examples are for illustrative purposes only and are not to be construed as limiting the invention.

### DESCRIPTION OF THE FIGURES

FIG. 1: Plasmid map of pGBS416ICL-1, encoding isocitrate lyase (ICL1) from K. lactis for expression in S. cerevisiae. CPO denotes codon pair optimized.

FIG. 2: Plasmid map of pGBS416ICL-2, encoding isocitrate lyase (ICL1) from K. lactis and malate synthase (MLS1) from S. cerevisiae for expression in S. cerevisiae. CPO denotes codon pair optimized.

FIG. 3: Plasmid map of pGBS414PEK-2, containing PEP carboxykinase from Mannheimia succiniciproducens (PCKm) and mitochondrial fumarate reductase m1 from Trypanosoma brucei (FRDm1) for expression in Saccharomyces cerevisiae. The synthetic gene constructs TDH1 promoter-PCKm-TDH1 terminator and TDH3 promoter-FRDm1-TDH3 terminator were cloned into expression vector pRS414. CPO denotes codon pair optimized.

FIG. 4: Plasmid map of pGBS415FUM-2, containing fumarase from Rhizopus oryzae (FUMR) and cytoplasmic malate dehydrogenase from Saccharomyces cerevisiae truncated for the first 12 amino acids (delta12N MDH2) for expression in Saccharomyces cerevisiae. The synthetic gene constructs TDH1 promoter-FUMR-TDH1 terminator and TDH3 promoter-delta12N MDH2-TDH3 terminator were cloned into expression vector pRS415. CPO denotes codon pair optimized.

### EXAMPLES

### Example 1A

### Cloning of Isocitrate Lyase From K. lactis and Malate Synthase From Saccharomyces cerevisiae in Saccharomyces cerevisiae and Production of Dicarboxylic Acid

### 1A.1. Expression Constructs

Isocitrate lyase [E.C. 4.2.1.2], GenBank accession number 21724726, from Kluyveromyces lactis and malate synthase [E.C. 2.3.3.9], GenBank accession number 3964, from Saccharomyces cerevisiae were analysed for the presence of signal sequences using SignalP 3.0 (http://www.cbs.dtu.dk/services/SignalP/) Bendtsen, J. et al. (2004) Mol. Biol., 340:783-795 and TargetP 1.1 (http://www.cbs.dtu.dk/services/TargetP/) Emanuelsson, O. et al. (2007) Nature Protocols 2, 953-971. No targeting sequences were identified for isocitrate lyase from K. lactis, a putative 3 amino acid peroxisomal targeting sequence was identified at the C-terminus of malate synthase of S. cerevisiae.

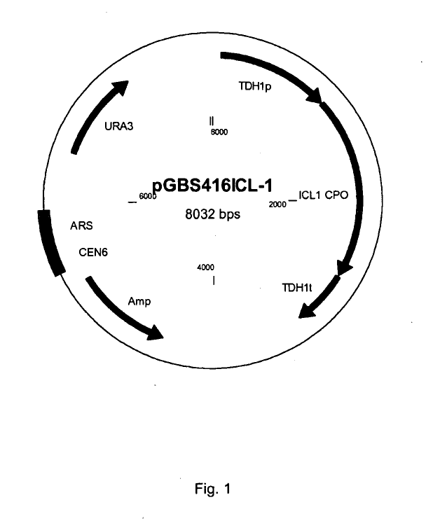

SEQ ID NO: 1 was subjected to the codon-pair method as disclosed in WO2008/000632 for S. cerevisiae. The resulting sequence SEQ ID NO: 6 was put behind the constitutive TDH1 promoter sequence SEQ ID NO: 8 and before the TDH1 terminator sequence SEQ ID NO: 9, and convenient restriction sites were added. SEQ ID NO: 5, lacking the peroxisomal targeting signal was subjected to the codon-pair method as disclosed in WO2008/000632 for S. cerevisiae. The resulting sequence SEQ ID NO: 7 was put behind the constitutive TDH3 promoter sequence SEQ ID NO: 10 and before the TDH3 terminator sequence SEQ ID NO: 11, and convenient restriction sites were added. The resulting sequences were synthesised at Sloning (Puchheim, Germany). The expression construct pGBS416ICL-2 was created after a BamHI/NotI restriction of the S. cerevisiae expression vector pRS416 (Sirkoski R. S. and Hieter P, Genetics, 1989, 122(1):19-27) and subsequently ligating in this vector by a 3-point ligation a BamHI/Ascl restricted fragment consisting of the isocitrate lyase (origin K. lactis) synthetic gene construct and an Ascl/NotI restricted fragment consisting of the malate synthase (origin S. cerevisiae) synthetic gene construct (FIG. 1). The ligation mixture is used for transformation of E. coli DH10B (Invitrogen) resulting in the yeast expression construct pGBS416ICL-2 (FIG. 1).

The construct pGBS416ICL-2 is transformed into S. cerevisiae strains CEN.PK113-6B (MATA ura3-52 leu2-112 trp1-289), RWB066 (MATA ura3-52 leu2-112 trp1-289 adh1::lox adh2::Kanlox) and RWB064 (MATA ura3-52 leu2-112 trp1-289 adh1::lox adh2::lox gpd1::Kanlox). Transformation mixtures are plated on Yeast Nitrogen Base (YNB) w/o AA (Difco)+2% glucose supplemented with appropriate amino acids. Transformants are inoculated in Verduyn medium containing 4% glucose supplemented with appropriate amino acids (Verduyn et al., 1992, Yeast. July; 8(7):501-17) and CaCO₃ and grown under aerobic, anaerobic and oxygen-limited conditions in shake flasks. The medium for anaerobic cultivation is supplemented with 0.01 g/l ergosterol and 0.42 g/l Tween 80 dissolved in ethanol (Andreasen and Stier, 1953, J. cell. Physiol, 41, 23-36; Andreasen and Stier, 1954, J. Cell. Physiol, 43: 271-281). All yeast cultures are grown at 30° C. in a shaking incubator at 250-280 rpm. At different incubation times, aliquots of the cultures are removed, centrifuged and the medium is analysed by HPLC for formation of oxalic acid, malic acid, fumaric acid and succinic acid as described below.

### 1A.2. HPLC Analysis

HPLC is performed for the determination of organic acids and sugars in different kinds of samples. The principle of the separation on a Phenomenex Rezex-RHM-Monosaccharide column is based on size exclusion, ion-exclusion and ion-exchange using reversed phase mechanisms. Detection takes place by differential refractive index and ultra violet detectors.

### Example 1B

### Cloning of Isocitrate Lyase From K. lactis and Malate Synthase From Saccharomyces cerevisiae in Saccharomyces cerevisiae and Production of Dicarboxylic Acid

### 1B.1. Expression Constructs

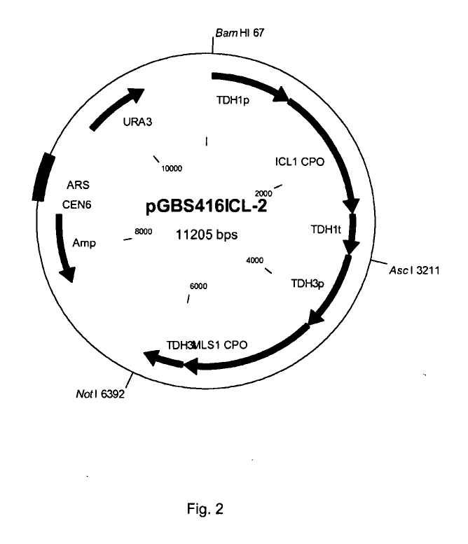

Potential targeting sequences of isocitrate lyase (ICL1 from K. lactis) or malate synthase (MLS1 from S. cerevisiae) were identified as disclosed in Example 1A.1. SEQ ID NO 1: was subjected to the codon-pair method as disclosed in WO2008/000632 for S. cerevisiae. The resulting sequence SEQ ID NO: 6 was put behind the constitutive TDH1 promoter sequence SEQ ID NO: 8 and before the TDH1 terminator sequence SEQ ID NO: 9, and convenient restriction sites were added. The resulting synthetic construct SEQ ID NO: 12 was synthesised at Sloning (Puchheim, Germany). SEQ ID NO: 5 was subjected to the codon-pair method as disclosed in WO2008/000632 for S. cerevisiae. The resulting sequence SEQ ID NO: 7 was put behind the constitutive TDH3 promoter sequence SEQ ID NO: 10 and before the TDH3 terminator sequence SEQ ID NO: 11, and convenient restriction sites were added. The resulting synthetic construct SEQ ID NO: 13 was synthesised at Sloning (Puchheim, Germany). The expression construct pGBS416ICL-1 was created after a BamHI/NotI restriction of the S. cerevisiae expression vector pRS416 (Sirkoski R. S. and Hieter P, Genetics, 1989, 122(1):19-27) and subsequently ligating in this vector a BamHI/NotI restriction fragment consisting of the isocitrate lyase (origin Kluveromyces lactis) synthetic gene construct (SEQ ID NO: 12). The ligation mixture was used for transformation of E. coli TOP10 (Invitrogen) resulting in the yeast expression construct pGBS416ICL-1 (FIG. 1). To create pGBS416ICL-2, pGBK416ICL-1 was restricted with Ascl and NotI. Subsequently, an Ascl/NotI restriction fragment consisting of MLS1 (origin S. cerevisiae) synthetic gene construct (SEQ ID NO: 13) was ligated into the restricted pGBS416ICL-1 vector, resulting in expression construct pGBS416ICL-2 (FIG. 2).

### 1B.2. Transformation

The constructs pGBS416ICL-1 and pGBS416ICL-2 were transformed into S. cerevisiae strain CEN.PK113-5D (MATA ura3-52), resulting in strains SUC-121 and SUC-122. As negative control, empty vector pRS416 was transformed into strain CEN.PK 113-5D, resulting in strain SUC-123. Transformation mixtures were plated on Yeast Nitrogen Base (YNB) w/o AA (Difco)+2% glucose.

### 1B.3. Growth Experiments

Transformants were inoculated in 20 ml pre-culture medium consisting of Verduyn medium (Verduyn et al., 1992, Yeast. July; 8(7):501-17) comprising 2% glucose (w/v) and grown under aerobic conditions in 100 ml shake flasks in a shaking incubator at 30° C. at 250 rpm. After 72 hours, the culture was centrifuged for 5 minutes at 4750 rpm. The supernatant was decanted and the pellet (cells) was resuspended in production medium. The production medium consisted of Verduyn medium with 10% glucose (w/v) and 1% CaCO₃ (w/v). The cells were grown in 50 ml production medium in 100 ml shake flasks in a shaking incubator at 30° C. at 100 rpm. After 4 and 7 days incubation, a 1 ml sample was taken from the culture and succinic acid levels were measured by HPLC as described in section 1A.2. The results are shown in Table 1.

TABLE 1
Effect of introduction of isocitrate lyase from K. lactis and malate
synthase from S. cerevisiae in S. cerevisiae on succinic acid
production levels after 4 and 7 days of incubation in shake flask.
Results are the average of 3 individual growth experiments.

| S. cerevisiae | Over- | Succinic | Succinic |
|---|---|---|---|
| strain comprising | expressed | acid (mg/l) | acid (mg/l) |
| plasmid: | genes | after 4 days | after 7 days |
| pGBS416ICL-1 | ICL1 | 399 ± 6 | 460 ± 11 |
| (SUC-121) | | | |
| pGBS416ICL-2 | ICL1, MLS1 | 420 ± 24 | 477 ± 36 |
| (SUC-122) | | | |
| pRS416 (empty) | — | 332 ± 20 | 394 ± 22 |
| (SUC-123) | | | |

The results in Table 1 show that introduction and overexpression of isocitrate lyase (ICL1) from K. lactis resulted in increased succinic acid production levels (1.20 fold after 4 days incubation and 1.17 fold after 7 days compared to the empty vector control strain). Furthermore, introduction and overexpression of isocitrate lyase (ICL1) from K. lactis and additional overexpression of malate synthase (MLS1) from S. cerevisiae resulted in increased succinic acid production levels (1.27 fold after 4 days incubation and 1.21 fold after 7 days compared to the empty vector control strain).

### Example 1C

### Expression Isocitrate Lyase From Kluyveromyces lactis and Malate Synthase From Saccharomyces cerevisiae in Addition to PEP Carboxykinase From Mannheimia succiniciproducens and Malate Dehydrogenase from Saccharomyces cerevisiae and Fumarase from Rhizopus oryzae and Fumarate Reductase From Trypanosoma brucei in Saccharomyces cerevisiae

### 1C.1. Gene Sequences

### Phosphoenolpyruvate Carboxykinase:

Phosphoenolpyruvate carboxykinase [E.C. 4.1.1.49], GenBank accession number 52426348, from Mannheimia succiniciproducens was analysed for the presence of signal sequences as described in Schlüter et al., (2007) NAR, 35, D815-D822. The sequence as shown in SEQ ID NO: 14 required no modifications. SEQ ID NO: 14 was subjected to the codon-pair method as disclosed in WO2008/000632 for S. cerevisiae. The stop codon TAA in the resulting sequence SEQ ID NO: 15 was modified to TAAG. SEQ ID NO: 15 containing stop codon TAAG was put behind the constitutive TDH1 promoter sequence SEQ ID NO: 8 and before the TDH1 terminator sequence SEQ ID NO: 9. Convenient restriction sites were added. The resulting synthetic construct (SEQ ID NO: 16) was synthesised at Stoning (Puchheim, Germany).

### Malate Dehydrogenase

Cytoplasmic malate dehydrogenase (Mdh2p) [E.C. 1.1.1.37], GenBank accession number 171915, is regulated by carbon catabolite repression: transcription of MDH2 is repressed and Mdh2p is degraded upon addition of glucose to glucose-starved cells. Mdh2p deleted for the 12 amino-terminal amino acids is less-susceptible for glucose-induced degradation (Minard and McAlister-Henn, J Biol. Chem. 1992 Aug. 25; 267(24):17458-64). To avoid glucose-induced degradation of Mdh2, the nucleotides encoding the first 12 amino acids were removed, and a new methionine amino acid was introduced (SEQ ID NO: 17) for overexpression of Mdh2 in S. cerevisiae. SEQ ID NO: 17 was subjected to the codon-pair method as disclosed in WO2008/000632 for S. cerevisiae. The stop codon TAA in the resulting in SEQ ID NO: 18, was modified to TAAG. SEQ ID NO: 18 containing a modified stop codon TAAG, encoding delta12NMDH2, was put behind the constitutive TDH3 promoter sequence SEQ ID NO: 10 and before the TDH3 terminator sequence SEQ ID NO: 11, and convenient restriction sites were added. The resulting synthetic construct (SEQ ID NO: 19) was synthesised at Sloning (Puchheim, Germany).

### Fumarase:

Fumarase [E.C. 4.2.1.2], GenBank accession number 469103, from Rhizopus oryzae (FumR) was analysed for the presence of signal sequences using SignalP 3.0 (http://www.cbs.dtu.dk/services/SignalP/) Bendtsen, J. et al. (2004) Mol. Biol., 340:783-795 and TargetP 1.1 (http://www.cbs.dtu.dk/services/TargetP/) Emanuelsson, O. et al. (2007) Nature Protocols 2, 953-971. A putative mitochondrial targeting sequence in the first 23 amino acid of the protein was identified. To avoid potential targeting to mitochondria in S. cerevisiae, the first 23 amino acids were removed from FumR and a methionine amino acid was reintroduced resulting in SEQ ID NO: 20. SEQ ID NO: 20 was subjected to the codon-pair method as disclosed in WO2008/000632 for S. cerevisiae resulting in SEQ ID NO: 21. The stop codon TAA in SEQ ID NO: 21 was modified to TAAG. SEQ ID NO: 21 containing TAAG as stop codon was synthesized behind the constitutive TDH1 promoter sequence SEQ ID NO: 8 and before the TDH1 terminator sequence SEQ ID NO: 9 and convenient restriction sites were added. The resulting synthetic construct SEQ ID NO: 22 was synthesised at Sloning (Puchheim, Germany).

### Fumarate Reductase:

Mitochondrial fumarate reductase m1 (FRDm1) [E.C. 1.3.1.6], GenBank accession number 60460035, from Trypanosoma brucei was analysed for the presence of signal sequences using SignalP 3.0 (http://www.cbs.dtu.dk/services/SignalP/) Bendtsen, J. et al. (2004) Mol. Biol., 340:783-795 and TargetP 1.1 (http://www.cbs.dtu.dk/services/TargetP) Emanuelsson, O. et al. (2007) Nature Protocols 2, 953-971. A putative mitochondrial targeting sequence in the N-terminal half of the protein was identified, including a possible cleavage site between pos. 25 and 26 (D-S).

It was shown that FRDm1 recombinant protein lacking the 68 N-terminal residues, relocalized to the cytosol of the procyclic trypanosomes (Coustou et al., J Biol. Chem. 2005 Apr. 29; 280(17):16559-70). These results indicate that the predicted N-terminal signal motif of FRDm1 is required for targeting to the mitochondrion. To avoid potential targeting to mitochondria in S. cerevisiae, the first 68 amino acids were removed from the mitochondrial fumarate reductase amino acid sequence and a methionine amino acid was reintroduced resulting in SEQ ID NO: 23. SEQ ID NO: 23 codon optimized as disclosed in WO2008/000632 for expression in S. cerevisiae. The stop codon TGA in SEQ ID NO: 24 was modified to TAAG. The resulting sequence SEQ ID NO: 24 was put behind the constitutive TDH3 promoter sequence SEQ ID NO: 10 and before the TDH3 terminator sequence SEQ ID NO: 11, and convenient restriction sites were added. The resulting synthetic construct SEQ ID NO: 25 was synthesised at Sloning (Puchheim, Germany).

### 1C.2. Construction of Expression Constructs

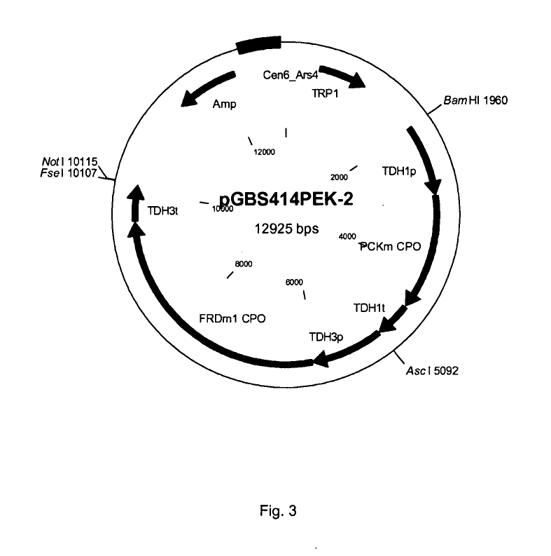

The expression construct pGBS414PEK-2 (FIG. 3) was created after a BamHI/NotI restriction of the S. cerevisiae expression vector pRS414 (Sirkoski R. S. and Hieter P, Genetics, 1989, 122(1):19-27) and subsequently ligating in this vector a BamHI/NotI restriction fragment consisting of the phosphoenolpyruvate carboxykinase (origin Mannheimia succiniciproducens) synthetic gene construct (SEQ ID NO: 16). The ligation mix was used for transformation of E. coli TOP10 (Invitrogen) resulting in the yeast expression construct pGBS414PEK-1. Subsequently, pGBK414PEK-1 was restricted with Ascl and NotI. To create pGBS414PEK-2, an Ascl/NotI restriction fragment consisting of mitochondrial fumarate reductase from T. brucei (FRDm1) synthetic gene construct (SEQ ID NO: 25) was ligated into the restricted pGBS414PEK-1 vector. The ligation mix was used for transformation of E. coli TOP10 (Invitrogen) resulting in the yeast expression construct pGBS414PEK-2 (FIG. 3).

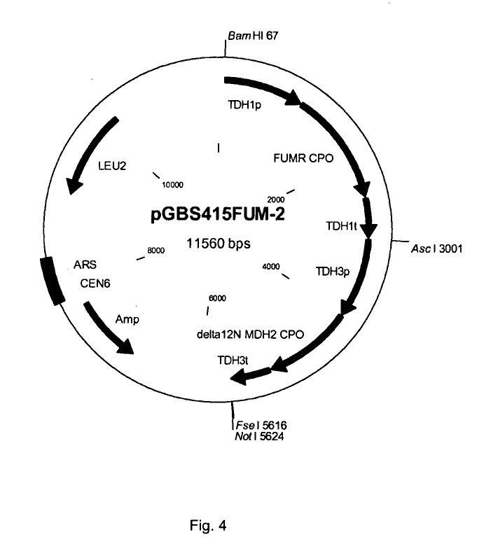

The expression construct pGBS415FUM-2 (FIG. 4) was created after a BamHI/NotI restriction of the S. cerevisiae expression vector pRS415 (Sirkoski R. S. and Hieter P, Genetics, 1989, 122(1):19-27) and subsequently ligating in this vector a BamHI/NotI restriction fragment consisting of the fumarase (origin Rhizopus oryzae) synthetic gene construct (SEQ ID NO: 22). The ligation mix was used for transformation of E. coli TOP10 (Invitrogen) resulting in the yeast expression construct pGBS415FUM-1. Subsequently, pGBK415FUM-1 was restricted with Ascl and NotI. To create pGBS415FUM-2, an Ascl/NotI restriction fragment consisting of cytoplasmic malate dehydrogenase from S. cerevisiae (delta12N MDH2) synthetic gene construct (SEQ ID NO: 19) was ligated into the restricted pGBS415FUM-1 vector. The ligation mix was used for transformation of E. coli TOP10 (Invitrogen) resulting in the yeast expression construct pGBS415FUM-2 (FIG. 4).

1C.3. S. cerevisiae Strains

Plasmids pGBS414PEK-2, pGBS415FUM-2 and pGBS416ICL-2 or pRS416 were transformed into S. cerevisiae strain CEN.PK113-6B (MATA ura3-52 leu2-112 trp1-289), resulting in the yeast strains depicted in Table 2. An empty vector control strain was created by transformation of pRS414, pRS415 and pRS416 empty vectors (Sirkoski R. S. and Hieter P, Genetics, 1989, 122(1):19-27). The expression vectors were transformed into yeast by electroporation. The transformation mixtures were plated on Yeast Nitrogen Base (YNB) w/o AA (Difco)+2% glucose.

TABLE 2
Yeast strains constructed for Example 1C.

| Name | Background | Plasmids | Genes |
|---|---|---|---|
| SUC-131 | CEN.PK113-6B | pGBS414PEK-2 | PCKm, FRDm1 |
| | | pGBS415FUM-2 | FUMR, delta12N MDH2 |
| | | pRS416ICL-2 | ICL1, MLS1 |
| SUC-132 | CEN.PK113-6B | pGBS414PEK-2 | PCKm, FRDm1 |
| | | pGBS415FUM-2 | FUMR, delta12N MDH2 |
| | | pRS416 | |
| | | (empty vector) | |
| SUC-101 | CEN.PK113-6B | pRS414 | |
| | | (empty vector) | |
| | | pRS415 | |
| | | (empty vector) | |
| | | pRS416 | |
| | | (empty vector) | |

### 1C.4. Growth Experiments and Succinic Acid Production

Transformants were grown and samples were taken as described in section 1B.3. Succinic acid levels were measured by HPLC as described in section 1A.2. The results are shown in Table 3.

TABLE 3
Effect of introduction of isocitrate lyase from Kluyveromyces
lactis and malate synthase from Saccharomyces cerevisiae in
addition to PEP carboxykinase from Mannheimia succiniciproducens
and malate dehydrogenase from Saccharomyces cerevisiae and
fumarase from Rhizopus oryzae and fumarate reductase from
Trypanosoma brucei on succinic acid production in
Saccharomyces cerevisiae. Data represent values as
measured in supernatant of cells grown in shake flask cultures.
The number of individual growth experiments is indicated.

| | Succinic acid (g/l) | Succinic acid (g/l) |
|---|---|---|
| Strain: | after 4 days | after 7 days |
| SUC-131 (ICL1, MLS1) | 6.02 ± 0.27 (n = 3) | 6.34 ± 0.16 (n = 3) |
| SUC-132 (control) | 5.76 ± 0.32 (n = 3) | 5.90 ± 0.30 (n = 3) |
| SUC-101 (empty vector | 0.34 ± 0.01 (n = 6) | 0.34 ± 0.02 (n = 6) |
| control) | | |

The results in Table 3 show that introduction and overexpression PEP carboxykinase from Mannheimia succiniciproducens and malate dehydrogenase from Saccharomyces cerevisiae and fumarase from Rhizopus oryzae and fumarate reductase from Trypanosoma brucei results in increased succinic acid production in Saccharomyces cerevisiae, as compared to a strain modified with an empty vector (SUC-101, approximately 17 fold increase compared to empty vector control after 4 and 7 days of growth). Expression of isocitrate lyase from K. lactis and malate synthase from S. cerevisiae in addition to PCKm, delta12N MDH2, FUMR and FRDm1 resulted in a further increase in succinic acid production levels (1.05 fold after 4 and 1.07 fold increase after 7 days of growth), thus showing a positive effect of the glyoxylate cycle on succinic acid production in S. cerevisiae.

1. Process for the production of a dicarboxylic acid comprising fermenting a eukaryotic cell in a suitable fermentation medium, wherein the eukaryotic cell comprises an enzyme which catalyses the conversion of isocitric acid to succinic acid, and producing the dicarboxylic acid, wherein succinic acid is produced in the cytosol.

2. Process according to claim 1, wherein the enzyme is an isocitrate lyase.

3. Process according to claim 1 wherein the enzyme has at least 30% sequence identity with the amino acid sequence of SEQ ID NO: 1.

4. Process for the production of a dicarboxylic acid, optionally according to claim 1, wherein the eukaryotic cell comprises an enzyme which catalyses the conversion of glyoxylic acid to malic acid, wherein malic acid is produced in the cytosol.

5. Process according to claim 4, wherein the enzyme is a malate synthase.

6. Process according to claim 4, wherein the enzyme has at least 40% sequence identity with the amino acid sequence of SEQ ID NO: 5.

7. Process according to claim 1, wherein the eukaryotic cell is a yeast or a filamentous fungus, selected from the group consisting of the genus Saccharomyces, Aspergillus, Penicillium, Pichia, Kluyveromyces, Yarrowia, Candida, Hansenula, Trichosporon, Trichoderma, Rhizopus, or Zygosaccharomyces.

8. Process according to claim 1, wherein the dicarboxylic acid is recovered from the fermentation broth, and optionally purified.

9. Process according to claim 1 further comprising recovering the dicarboxylic acid is malic acid, fumaric acid or succinic acid from the fermentation medium.

10. Process according to claim 1 comprising further using the dicarboxylic acid produced for the preparation of a pharmaceutical, cosmetic, food, feed or chemical product.

11. A eukaryotic cell comprising a nucleotide sequence encoding a first enzyme which catalyses the conversion of isocitric acid to succinic acid, and a nucleotide sequence encoding a second enzyme which catalyses the conversion of glyoxylic acid to malic acid, wherein the first and the second enzyme are active in the cytosol.

12. A eukaryotic cell according to claim 11, wherein the first enzyme is an isocitrate lyase.

13. A eukaryotic cell according to claim 11, wherein the second enzyme is a malate synthase.

14. A eukaryotic cell according to claim 11, wherein the cell is a yeast.

15. A eukaryotic cell according to claim 11, which is a Saccharomyces cerevisiae comprising a nucleotide sequence of SEQ ID NO: 6 encoding an enzyme having isocitrate lyase activity and a nucleotide sequence of SEQ ID NO: 7 encoding an enzyme having malate synthase activity.

16. A eukaryotic cell transformed such that the cell is capable of producing a dicarboxylic acid by fermenting the cell in a suitable fermentation medium wherein the cell comprises an enzyme catalysing the conversion of isocitric acid to succinic acid, wherein succinic acid is produced in the cytosol and/or an enzyme that catalyses the conversion of glyoxylic acid to malic acid, wherein malic acid is produced in the cytosol.


SEQUENCE LISTING (25 sequences)

SEQ ID NO: 1 - protein, 542 aa, Kluyveromyces lactis
MVSVKASAAEKKEFLQSQIDEIEKWWSEPRWKDTKRIYSAYEIAKRRGSVKPNTFPSTVMSQKLFKILGEHAKNGTVSKTFGALDPVQVTQMSKYLDTIYVSGWQCSSTASTSNEPGPDLADYPMDTVPNKVEHLFKAQQFHDRKQWERICDGTIEESEIIDYLTPIVADGDAGHGGLTAVFKLTKMFIERGAAGIHIEDQTSTNKKCGHMAGRCVIPVQEHINRLITCRMAADVLGSDLILVARTDSEAATLLSSTADSRDHYFILGASNPAVKGKPLNDLLNKAILDGATIDDLQTIEKEWLAKADVKLFHEVFADAAKAAGKDQSVIDQFNSKVNPLSETSIYEMQALAKELLGTELFFDWDLPRGREGLYRYQGGTQCSVMRARAFAPYADLCWMESNYPDYEQAKEFAEGVTAKFPGKWMAYNLSPSFNWTKAMSVDEQETFIQRLGDLGYIWQFITLAGLHTSGLAIEQFSKNFAKLGMKAYAQDIQKKELDNGIDMVKHQKWSGAEYIDGLLRLAQGGLAATAAMGQGVTEDQFK

SEQ ID NO: 2 - DNA, 1629 bp, Kluyveromyces lactis
atggtctccgttaaggcttctgctgctgaaaagaaggaattcttacaaagtcaaattgacgaaattgagaaatggtggtccgaacctcgttggaaggacaccaaacgtatctactctgcttacgaaattgcaaagagacgtggttcagtaaaaccaaatactttcccatctactgtgatgtctcaaaaattgttcaaaatcttgggggaacacgctaagaacggcactgtctccaagactttcggtgctttggaccctgttcaagtgacccaaatgtctaagtacttggataccatttacgtttcaggctggcaatgttcttccactgcatcaacttctaacgaaccgggtccagatttggctgattacccaatggacactgtaccaaacaaagtggagcatttgttcaaggctcaacaattccatgacagaaaacaatgggaacgtatctgtgatggtaccattgaagaatctgaaatcattgactatttgactccaatcgttgctgatggtgacgctggtcatggtggtttgactgctgtcttcaagttgacaaaaatgttcatcgaaagaggagctgccggtatccacattgaagatcaaacttctacaaacaaaaagtgtggtcacatggctggtagatgtgttatcccagtacaagaacatattaacagattgatcacttgtagaatggctgcagatgttttgggttcagatttgatccttgttgccagaactgattctgaagccgctactctattatcctctactgccgattcaagggaccattacttcatcttgggtgcttccaatccagccgtcaagggtaaaccattgaacgatttactgaacaaagctatcttggacggtgccaccatcgatgacttacaaaccatcgaaaaagaatggttggccaaggccgacgttaagctgttccacgaagtattcgctgacgctgccaaggctgcaggtaaggaccaatccgtaattgaccaattcaacagcaaagtcaacccattaagtgaaacatcaatctatgaaatgcaagccttggccaaggaattgttgggtaccgaattgttcttcgactgggacttgccaagaggccgtgaaggtttgtaccgttaccaaggtggtactcagtgttctgtgatgagagctcgtgcctttgctccatacgctgatctatgttggatggaatctaactaccctgattacgaacaagctaaggaattcgctgaaggtgtcacagctaagttcccaggcaagtggatggcttacaacttgtcaccatctttcaactggacaaaagctatgtctgtcgacgaacaagagacattcattcaaagattgggtgacttgggttacatctggcaatttatcactttagctggtctacatacaagtggtttagccatcgaacaattctccaagaatttcgccaagttaggaatgaaggcatatgctcaagatatccaaaagaaggaattggataacggtatcgacatggtaaagcaccagaaatggtctggtgctgaatatatcgatggtctattaagattagcacaaggtggtctcgctgccactgctgctatgggtcaaggtgtcactgaggatcagttcaaataa

SEQ ID NO: 3 - protein, 554 aa, Saccharomyces cerevisiae
MVKVSLDNVKLLVDVDKEPFFKPSSTTVGDILTKDALEFIVLLHRTFNNKRKQLLENRQVVQKKLDSGSYHLDFLPETANIRNDPTWQGPILAPGLINRSTEITGPPLRNMLINALNAPVNTYMTDFEDSASPTWNNMVYGQVNLYDAIRNQIDFDTPRKSYKLNGNVANLPTIIVRPRGWHMVEKHLYVDDEPISASIFDFGLYFYHNAKELIKLGKGPYFYLPKMEHHLEAKLWNDVFCVAQDYIGIPRGTIRATVLIETLPAAFQMEEIIYQLRQHSSGLNCGRWDYIFSTIKRLRNDPNHILPNRNQVTMTSPFMDAYVKRLINTCHRRGVHAMGGMAAQIPIKDDPAANEKAMTKVRNDKIRELTNGHDGSWVAHPALAPICNEVFINMGTPNQIYFIPENVVTAANLLETKIPNGEITTEGIVQNLDIGLQYMEAWLRGSGCVPINNLMEDAATAEVSRCQLYQWVKHGVTLKDTGEKVTPELTEKILKEQVERLSKASPLGDKNKFALAAKYFLPEIRGEKFSEFLTTLLYDEIVSTKATPTDLSKL

SEQ ID NO: 4 - DNA, 1665 bp, Saccharomyces cerevisiae
atggttaaggtcagtttggataacgtcaaattactggtggatgttgataaggagcctttctttaaaccatctagtactacagtgggagatattcttaccaaggatgctctagagttcattgttcttttacacagaactttcaacaacaagagaaaacaattattggaaaacagacaagttgttcagaagaaattagactcgggctcctatcatctggatttcctgcctgaaactgcaaatattagaaatgatcccacttggcaaggtccaattttggcaccggggttaattaataggtcaacggaaatcacagggcctccattgagaaatatgctgatcaacgctttgaatgctcctgtgaacacctatatgactgattttgaagattcagcttcacctacttggaacaacatggtttacggtcaagttaatctctacgacgcgatcagaaatcaaatcgattttgacacaccaagaaaatcgtacaaattgaatggaaatgtggccaacttgcccactattatcgtgagaccccgtggttggcacatggtggaaaagcacctttatgtagatgatgaaccaatcagcgcttccatctttgattttggtttatatttctaccataatgccaaagaattaatcaaattgggcaaaggtccttacttctatttgccaaagatggagcaccacttggaagctaaactatggaacgacgtcttctgtgtagctcaagattacattgggatcccaaggggtacaatcagagctactgtgttgattgaaactttgcctgctgctttccaaatggaagagatcatctatcaattaagacaacattctagtgggttgaattgcggacgttgggactatattttctctacaatcaagagattaagaaatgatcctaatcacattttgcccaatagaaatcaagtgactatgacttccccattcatggatgcatacgtgaaaagattaatcaatacctgtcatcggaggggtgttcatgccatgggtggtatggctgcgcaaatccctatcaaagacgacccggcagccaatgaaaaggccatgactaaagtccgtaatgataagattagagagctgacaaatggacatgatgggtcatgggttgcacacccagcactggcccctatttgtaatgaagttttcattaatatgggaacaccaaaccaaatctatttcattcctgaaaacgttgtaacggctgctaatctgctggaaaccaaaattccaaatggtgagattactaccgagggaattgtacaaaacttggatatcgggttgcagtacatggaagcttggctcagaggctctggatgtgtgcccatcaacaacttgatggaagacgccgccactgctgaagtgtctcgttgtcaattgtatcaatgggtgaaacacggtgttactctaaaggacacgggagaaaaggtcaccccagaattaaccgaaaagattctaaaagaacaagtggaaagactgtctaaggcaagtccattgggtgacaagaacaaattcgcgctggccgctaagtatttcttgccagaaatcagaggcgagaaattcagtgaatttttgactacattgttgtacgacgaaattgtgtccactaaggcgacgcccactgatttgagcaaattgtga

SEQ ID NO: 5 - protein, 551 aa, Artificial sequence
  SEQ ID NO 5 Saccharomyces cerevisiae MLS 1 lacking 3 a.a. C terminal targeting signal
MVKVSLDNVKLLVDVDKEPFFKPSSTTVGDILTKDALEFIVLLHRTFNNKRKQLLENRQVVQKKLDSGSYHLDFLPETANIRNDPTWQGPILAPGLINRSTEITGPPLRNMLINALNAPVNTYMTDFEDSASPTWNNMVYGQVNLYDAIRNQIDFDTPRKSYKLNGNVANLPTIIVRPRGWHMVEKHLYVDDEPISASIFDFGLYFYHNAKELIKLGKGPYFYLPKMEHHLEAKLWNDVFCVAQDYIGIPRGTIRATVLIETLPAAFQMEEIIYQLRQHSSGLNCGRWDYIFSTIKRLRNDPNHILPNRNQVTMTSPFMDAYVKRLINTCHRRGVHAMGGMAAQIPIKDDPAANEKAMTKVRNDKIRELTNGHDGSWVAHPALAPICNEVFINMGTPNQIYFIPENVVTAANLLETKIPNGEITTEGIVQNLDIGLQYMEAWLRGSGCVPINNLMEDAATAEVSRCQLYQWVKHGVTLKDTGEKVTPELTEKILKEQVERLSKASPLGDKNKFALAAKYFLPEIRGEKFSEFLTTLLYDEIVSTKATPTDL

SEQ ID NO: 6 - DNA, 1629 bp, Artificial sequence
  SEQ ID NO 6 Nucleotide sequence codon pair optimized for S. cerevisiae encoding for K. lactis isocitrate lyase (Icl1). (Stop codon TAA is TAAT in ordered construct)
atggtttccgtcaaggcttctgctgctgaaaagaaggaattcttgcaatctcaaatcgatgaaattgaaaaatggtggtctgaaccaagatggaaggacaccaagagaatctactctgcttacgaaattgccaagcgtcgtggttctgtcaagccaaacactttcccatctaccgtcatgtctcaaaaattgttcaagatcttaggtgaacacgctaagaacggtactgtttccaagactttcggtgctttggaccctgttcaagtcactcaaatgtccaagtacttggacaccatctacgtttccggttggcaatgttcctctactgcttccacttctaacgaaccaggtccagatttggctgactacccaatggacaccgttccaaacaaggttgaacatttgttcaaggctcaacaattccacgacagaaagcaatgggaaagaatctgtgatggtaccattgaagaatctgaaatcattgactacttgactccaattgttgctgatggtgatgctggtcacggtggtttgactgctgtcttcaagttgaccaagatgttcatcgaaagaggtgctgctggtattcacattgaagatcaaacctctaccaacaagaaatgtggtcacatggctggtagatgtgtcattccagttcaagaacacatcaacagattaatcacctgtagaatggctgctgatgtcttgggttctgacttgatcttagtcgccagaactgactctgaagctgctactttgttgtcctccactgctgactctcgtgaccattatttcatcttaggtgcttccaacccagctgtcaagggtaagcctttgaatgacttgttgaacaaggccatcttggatggtgctaccatcgatgacttgcaaaccattgaaaaggaatggttagccaaggctgatgtcaaattattccacgaagttttcgctgatgctgccaaggctgctggtaaggaccaatctgtcattgaccaattcaactccaaggttaacccattgtctgaaacctccatctacgaaatgcaagctttggccaaggaattgttgggtactgaattgttcttcgactgggacttgccaagaggtagagaaggtctatacagataccaaggtggtactcaatgttctgttatgagagccagagcctttgctccatacgctgatctatgttggatggaatccaactacccagactacgaacaagccaaggaatttgctgaaggtgttaccgccaagttcccaggtaaatggatggcttacaacttgtctccatctttcaactggaccaaggccatgtctgttgacgaacaagaaactttcatccaaagattaggtgacttgggttacatctggcaattcatcactttggctggtttgcacacctctggtttggccattgaacaattctccaagaactttgccaaattgggtatgaaggcttacgctcaagatatccaaaagaaggaattggacaacggtattgacatggttaagcaccaaaaatggtccggtgctgaatacatcgatggtttgttgagattggctcaaggtggtttggctgctaccgctgccatgggtcaaggtgtcactgaagatcaattcaagtaa

SEQ ID NO: 7 - DNA, 1656 bp, Artificial sequence
  Nucleotide sequence codon pair optimized for S. cerevisiae encoding for S. cerevisiae malate synthase (Mls1), lacking 3 amino acid C-terminal targeting signal. (Stop codon TAA is TAAT in ordered construct)
atggtcaaggtttctttggacaatgtcaaattgttagtcgatgttgacaaggaacctttcttcaagccttcttccaccaccgttggtgacatcttgaccaaggatgctttggaattcattgtcttgttgcacagaactttcaacaacaagagaaagcaattgttggaaaacagacaagttgttcaaaagaaattggactctggttcttaccatttggacttcttgccagaaactgctaacatcagaaacgacccaacctggcaaggtccaattttggctccaggtttgatcaacagatccactgaaatcactggtcctccattgagaaacatgttgatcaatgctttgaatgctccagttaacacctacatgactgacttcgaagattctgcctctccaacctggaacaacatggtttacggtcaagtcaacttatacgatgctatcagaaaccaaattgacttcgacactccaagaaaatcttacaaattgaacggtaacgttgccaacttgccaaccattattgtcagaccaagaggttggcacatggttgaaaagcatttatacgttgacgacgaaccaatttctgcctccattttcgatttcggtctatatttctaccataacgctaaggaattgatcaagttgggtaagggtccatacttctacttgccaaagatggaacaccacttggaagctaagttgtggaacgatgttttctgtgttgctcaagactacattggtattccaagaggtaccatcagagctactgttttgattgaaactttaccagctgctttccaaatggaagaaatcatctaccaattgagacaacactcctctggtttgaactgtggtagatgggactacatcttttccaccatcaagagattgagaaacgacccaaaccacattttgccaaacagaaaccaagtcaccatgacttctccattcatggacgcttacgtcaagagattgatcaacacctgtcaccgtcgtggtgtccacgctatgggtggtatggctgctcaaattccaatcaaggatgacccagctgccaacgaaaaggccatgaccaaggtcagaaacgacaagatcagagaattaaccaacggtcacgatggttcctgggttgctcacccagctttggctccaatctgtaacgaagtctttatcaacatgggtactccaaaccaaatctacttcattccagaaaacgttgtcactgctgctaacttgttggaaaccaagattccaaacggtgaaatcaccactgaaggtattgtccaaaacttggatatcggtttgcaatacatggaagcttggttacgtggttctggttgtgttccaatcaacaacttgatggaagatgccgctactgctgaagtttcccgttgtcaattgtaccaatgggttaagcacggtgtcactttgaaagacaccggtgaaaaggtcactccagaattgactgaaaagatcttaaaggaacaagttgaaagattatccaaagcctctccattaggtgacaagaacaagttcgctctagccgccaaatacttcttgccagaaatcagaggtgaaaagttctctgaatttttgaccactttgttgtacgatgaaattgtctccaccaaggctactccaaccgatttgtaa

SEQ ID NO: 8 - DNA, 1000 bp, Artificial sequence
  Sequence of the TDH1 promoter (Saccharomyces cerevisiae)
cttcccttttacagtgcttcggaaaagcacagcgttgtccaagggaacaatttttcttcaagttaatgcataagaaatatctttttttatgtttagctaagtaaaagcagcttggagtaaaaaaaaaaatgagtaaatttctcgatggattagtttctcacaggtaacataacaaaaaccaagaaaagcccgcttctgaaaactacagttgacttgtatgctaaagggccagactaatgggaggagaaaaagaaacgaatgtatatgctcatttacactctatatcaccatatggaggataagttgggctgagcttctgatccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaa

SEQ ID NO: 9 - DNA, 500 bp, Artificial sequence
  Sequence of the TDH1 terminator (Saccharomyces cerevisiae)
ataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgcctttgcaaaacgcacatacccacaatatgtgactattggcaaagaacgcattatcctttgaagaggtggatactgatactaagagagtctctattccggctccacttttagtccagagattacttgtcttcttacgtatcagaacaagaaagcatttccaaagtaattgcatttgcccttgagcagtatatatatactaagaa

SEQ ID NO: 10 - DNA, 1000 bp, Artificial sequence
  Sequence of the TDH3 promoter (Saccharomyces cerevisiae)
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 11 - DNA, 500 bp, Artificial sequence
  Sequence of the TDH3 terminator (Saccharomyces cerevisiae)
gtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagatgcagtaatatacacagattcc

SEQ ID NO: 12 - DNA, 3160 bp, Artificial sequence
  Synthetic construct TDH1p-ICL1-TDH1t for expression in S. cerevisiae
ggatcccttcccttttacagtgcttcggaaaagcacagcgttgtccaagggaacaatttttcttcaagttaatgcataagaaatatctttttttatgtttagctaagtaaaagcagcttggagtaaaaaaaaaaatgagtaaatttctcgatggattagtttctcacaggtaacataacaaaaaccaagaaaagcccgcttctgaaaactacagttgacttgtatgctaaagggccagactaatgggaggagaaaaagaaacgaatgtatatgctcatttacactctatatcaccatatggaggataagttgggctgagcttctgatccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaaatggtttccgtcaaggcttctgctgctgaaaagaaggaattcttgcaatctcaaatcgatgaaattgaaaaatggtggtctgaaccaagatggaaggacaccaagagaatctactctgcttacgaaattgccaagcgtcgtggttctgtcaagccaaacactttcccatctaccgtcatgtctcaaaaattgttcaagatcttaggtgaacacgctaagaacggtactgtttccaagactttcggtgctttggaccctgttcaagtcactcaaatgtccaagtacttggacaccatctacgtttccggttggcaatgttcctctactgcttccacttctaacgaaccaggtccagatttggctgactacccaatggacaccgttccaaacaaggttgaacatttgttcaaggctcaacaattccacgacagaaagcaatgggaaagaatctgtgatggtaccattgaagaatctgaaatcattgactacttgactccaattgttgctgatggtgatgctggtcacggtggtttgactgctgtcttcaagttgaccaagatgttcatcgaaagaggtgctgctggtattcacattgaagatcaaacctctaccaacaagaaatgtggtcacatggctggtagatgtgtcattccagttcaagaacacatcaacagattaatcacctgtagaatggctgctgatgtcttgggttctgacttgatcttagtcgccagaactgactctgaagctgctactttgttgtcctccactgctgactctcgtgaccattatttcatcttaggtgcttccaacccagctgtcaagggtaagcctttgaatgacttgttgaacaaggccatcttggatggtgctaccatcgatgacttgcaaaccattgaaaaggaatggttagccaaggctgatgtcaaattattccacgaagttttcgctgatgctgccaaggctgctggtaaggaccaatctgtcattgaccaattcaactccaaggttaacccattgtctgaaacctccatctacgaaatgcaagctttggccaaggaattgttgggtactgaattgttcttcgactgggacttgccaagaggtagagaaggtctatacagataccaaggtggtactcaatgttctgttatgagagccagagcctttgctccatacgctgatctatgttggatggaatccaactacccagactacgaacaagccaaggaatttgctgaaggtgttaccgccaagttcccaggtaaatggatggcttacaacttgtctccatctttcaactggaccaaggccatgtctgttgacgaacaagaaactttcatccaaagattaggtgacttgggttacatctggcaattcatcactttggctggtttgcacacctctggtttggccattgaacaattctccaagaactttgccaaattgggtatgaaggcttacgctcaagatatccaaaagaaggaattggacaacggtattgacatggttaagcaccaaaaatggtccggtgctgaatacatcgatggtttgttgagattggctcaaggtggtttggctgctaccgctgccatgggtcaaggtgtcactgaagatcaattcaagtaatgcccgggcataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgcctttgcaaaacgcacatacccacaatatgtgactattggcaaagaacgcattatcctttgaagaggtggatactgatactaagagagtctctattccggctccacttttagtccagagattacttgtcttcttacgtatcagaacaagaaagcatttccaaagtaattgcatttgcccttgagcagtatatatatactaagaaggcgcgccgcggccgc

SEQ ID NO: 13 - DNA, 3195 bp, Artificial sequence
  Synthetic construct TDH3p-MLS1-TDH3t for expression in S. cerevisiae
ggatccggcgcgccctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggtcaaggtttctttggacaatgtcaaattgttagtcgatgttgacaaggaacctttcttcaagccttcttccaccaccgttggtgacatcttgaccaaggatgctttggaattcattgtcttgttgcacagaactttcaacaacaagagaaagcaattgttggaaaacagacaagttgttcaaaagaaattggactctggttcttaccatttggacttcttgccagaaactgctaacatcagaaacgacccaacctggcaaggtccaattttggctccaggtttgatcaacagatccactgaaatcactggtcctccattgagaaacatgttgatcaatgctttgaatgctccagttaacacctacatgactgacttcgaagattctgcctctccaacctggaacaacatggtttacggtcaagtcaacttatacgatgctatcagaaaccaaattgacttcgacactccaagaaaatcttacaaattgaacggtaacgttgccaacttgccaaccattattgtcagaccaagaggttggcacatggttgaaaagcatttatacgttgacgacgaaccaatttctgcctccattttcgatttcggtctatatttctaccataacgctaaggaattgatcaagttgggtaagggtccatacttctacttgccaaagatggaacaccacttggaagctaagttgtggaacgatgttttctgtgttgctcaagactacattggtattccaagaggtaccatcagagctactgttttgattgaaactttaccagctgctttccaaatggaagaaatcatctaccaattgagacaacactcctctggtttgaactgtggtagatgggactacatcttttccaccatcaagagattgagaaacgacccaaaccacattttgccaaacagaaaccaagtcaccatgacttctccattcatggacgcttacgtcaagagattgatcaacacctgtcaccgtcgtggtgtccacgctatgggtggtatggctgctcaaattccaatcaaggatgacccagctgccaacgaaaaggccatgaccaaggtcagaaacgacaagatcagagaattaaccaacggtcacgatggttcctgggttgctcacccagctttggctccaatctgtaacgaagtctttatcaacatgggtactccaaaccaaatctacttcattccagaaaacgttgtcactgctgctaacttgttggaaaccaagattccaaacggtgaaatcaccactgaaggtattgtccaaaacttggatatcggtttgcaatacatggaagcttggttacgtggttctggttgtgttccaatcaacaacttgatggaagatgccgctactgctgaagtttcccgttgtcaattgtaccaatgggttaagcacggtgtcactttgaaagacaccggtgaaaaggtcactccagaattgactgaaaagatcttaaaggaacaagttgaaagattatccaaagcctctccattaggtgacaagaacaagttcgctctagccgccaaatacttcttgccagaaatcagaggtgaaaagttctctgaatttttgaccactttgttgtacgatgaaattgtctccaccaaggctactccaaccgatttgtaatgcccgggcgtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagatgcagtaatatacacagattccggccggccgcggccgc

SEQ ID NO: 14 - protein, 538 aa, Mannheimia succiniciproducens
MTDLNQLTQELGALGIHDVQEVVYNPSYELLFAEETKPGLEGYEKGTVTNQGAVAVNTGIFTGRSPKDKYIVLDDKTKDTVWWTSEKVKNDNKPMSQDTWNSLKGLVADQLSGKRLFVVDAFCGANKDTRLAVRVVTEVAWQAHFVTNMFIRPSAEELKGFKPDFVVMNGAKCTNPNWKEQGLNSENFVAFNITEGVQLIGGTWYGGEMKKGMFSMMNYFLPLRGIASMHCSANVGKDGDTAIFFGLSGTGKTTLSTDPKRQLIGDDEHGWDDEGVFNFEGGCYAKTINLSAENEPDIYGAIKRDALLENVVVLDNGDVDYADGSKTENTRVSYPIYHIQNIVKPVSKAGPATKVIFLSADAFGVLPPVSKLTPEQTKYYFLSGFTAKLAGTERGITEPTPTFSACFGAAFLSLHPTQYAEVLVKRMQESGAEAYLVNTGWNGTGKRISIKDTRGIIDAILDGSIDKAEMGSLPIFDFSIPKALPGVNPAILDPRDTYADKAQWEEKAQDLAGRFVKNFEKYTGTAEGQALVAAGPKA

SEQ ID NO: 15 - DNA, 1617 bp, Artificial sequence
  PEP carboxykinase M. succiniciproducens codon pair optimized for S. cerevisiae
atgaccgatttgaaccaattgactcaagaattgggtgctttgggtattcacgatgtccaagaagttgtctacaacccatcttacgaattgttgtttgctgaagaaaccaagccaggtttggaaggttacgaaaagggtactgttaccaaccaaggtgctgttgctgtcaacaccggtatcttcaccggtcgttctccaaaggacaaatacattgtcttggatgacaagaccaaggacactgtctggtggacttctgaaaaggtcaagaacgacaacaaaccaatgtcccaagacacttggaactctttaaagggtttagtcgctgaccaattgtctggtaagagattattcgttgtcgatgctttctgtggtgccaacaaggacaccagattagctgtcagagttgtcactgaagttgcttggcaagctcacttcgttaccaacatgttcatcagaccatctgctgaagaattgaaaggtttcaagccagatttcgttgtcatgaacggtgccaaatgtaccaacccaaactggaaggaacaaggtttgaactctgaaaactttgttgctttcaacatcactgaaggtgttcaattgattggtggtacctggtacggtggtgaaatgaagaagggtatgttctccatgatgaactacttcttgccattgagaggtattgcttccatgcactgttctgccaatgtcggtaaggacggtgacactgccatcttcttcggtctatccggtaccggtaagaccactttgtccactgacccaaagagacaattgattggtgatgacgaacacggttgggatgacgaaggtgttttcaactttgaaggtggttgttacgccaagaccatcaacttatctgctgaaaatgaaccagatatctacggtgccatcaagcgtgacgctctattggaaaacgttgttgttttggacaatggtgacgtcgattatgctgacggttccaagactgaaaacaccagagtttcttacccaatctaccatattcaaaacattgtcaagccagtttccaaggctggtccagctaccaaagttatcttcttgtctgctgatgctttcggtgttttgcctcctgtttccaagttgactccagaacaaaccaagtactacttcttgtctggtttcaccgccaagttggctggtactgaaagaggtatcactgaaccaactccaactttctctgcttgtttcggtgctgcctttttgtctttgcacccaactcaatacgctgaagttttggtcaagagaatgcaagaatctggtgctgaagcttacttggtcaacactggttggaacggtaccggtaagagaatctccatcaaagataccagaggtatcatcgatgccatcttggatggttccattgacaaggctgaaatgggttctttgccaattttcgatttctccattccaaaggctttgccaggtgtcaacccagccatcttagacccaagagacacctacgctgacaaagctcaatgggaagaaaaggctcaagacttggctggtagattcgtcaagaacttcgaaaaatacactggtactgctgaaggtcaagctttggttgctgctggtccaaaggcctaa

SEQ ID NO: 16 - DNA, 3148 bp, Artificial sequence
  Synthetic construct TDH1p-PCKm-TDH1t for expression in S. cerevisiae
ggatcccttcccttttacagtgcttcggaaaagcacagcgttgtccaagggaacaatttttcttcaagttaatgcataagaaatatctttttttatgtttagctaagtaaaagcagcttggagtaaaaaaaaaaatgagtaaatttctcgatggattagtttctcacaggtaacataacaaaaaccaagaaaagcccgcttctgaaaactacagttgacttgtatgctaaagggccagactaatgggaggagaaaaagaaacgaatgtatatgctcatttacactctatatcaccatatggaggataagttgggctgagcttctgatccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaaatgactgatttgaacaaattggtcaaggaattgaatgatttgggtttgactgacgtcaaggaaattgtctacaacccatcttacgaacaattattcgaagaagaaaccaagccaggtttggaaggtttcgacaagggtactttgaccactttaggtgctgttgctgttgacaccggtattttcaccggtcgttctccaaaggacaaatacattgtttgtgatgaaaccaccaaggacaccgtctggtggaactctgaagctgccaagaacgataacaagccaatgactcaagaaacctggaaatctttgagagaattggttgccaagcaattgtctggtaagagattattcgttgttgacgctttctgtggtgcttctgaaaagcacagaattggtgtcagaatggtcactgaagttgcttggcaagctcatttcgtcaagaacatgttcatcagaccaactgacgaagaattgaagaacttcaaggctgacttcaccgttttgaatggtgccaagtgtaccaacccaaactggaaggaacaaggtttgaactctgaaaactttgttgctttcaacatcactgaaggtatccaattgattggtggtacctggtacggtggtgaaatgaagaagggtatgttctccatgatgaactatttcttgccattgaaaggtgttgcttccatgcactgttctgccaatgtcggtaaggatggtgacgttgccatcttcttcggtctatccggtactggtaagaccactctatccactgacccaaagagacaattgattggtgatgacgaacacggttgggacgaatctggtgtctttaactttgaaggtggttgttacgccaagaccatcaacttatctcaagaaaacgaaccagatatctacggtgccatccgtcgtgatgctttgttggaaaacgttgttgtcagagctgacggttctgttgacttcgacgacggttccaagactgaaaacaccagagtttcttacccaatctaccacattgacaacattgtcagacctgtttccaaggctggtcacgctaccaaggttatcttcttgactgctgatgctttcggtgtcttgccacctgtttccaaattgactccagaacaaaccgaatactacttcttgtccggtttcactgccaaattggctggtactgaaagaggtgtcactgaaccaactccaactttctctgcttgtttcggtgctgctttcttatctttgcacccaatccaatacgctgatgtcttggttgaaagaatgaaggcttctggtgctgaagcttacttggtcaacaccggttggaacggtaccggtaagagaatctccatcaaggataccagaggtatcattgatgctatcttggacggttccattgaaaaggctgaaatgggtgaattgccaatcttcaacttggccattccaaaggctttgccaggtgttgacccagccatcttagatccaagagacacctacgctgacaaggctcaatggcaagtcaaggctgaagatttggctaacagattcgtcaagaactttgtcaaatacactgctaacccagaagctgccaaattggttggtgctggtccaaaggcttaaggcccgggcataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgcctttgcaaaacgcacatacccacaatatgtgactattggcaaagaacgcattatcctttgaagaggtggatactgatactaagagagtctctattccggctccacttttagtccagagattacttgtcttcttacgtatcagaacaagaaagcatttccaaagtaattgcatttgcccttgagcagtatatatatactaagaaggcgcgccgcggccgc

SEQ ID NO: 17 - protein, 365 aa, Artificial sequence
  MDH2 amino acid sequence from S. cerevisiae lacking first 12 a.a. + new methionine
MLKIAILGAAGGIGQSLSLLLKAQLQYQLKESNRSVTHIHLALYDVNQEAINGVTADLSHIDTPISVSSHSPAGGIENCLHNASIVVIPAGVPRKPGMTRDDLFNVNAGIISQLGDSIAECCDLSKVFVLVISNPVNSLVPVMVSNILKNHPQSRNSGIERRIMGVTKLDIVRASTFLREINIESGLTPRVNSMPDVPVIGGHSGETIIPLFSQSNFLSRLNEDQLKYLIHRVQYGGDEVVKAKNGKGSATLSMAHAGYKCVVQFVSLLLGNIEQIHGTYYVPLKDANNFPIAPGADQLLPLVDGADYFAIPLTITTKGVSYVDYDIVNRMNDMERNQMLPICVSQLKKNIDKGLEFVASRSASS

SEQ ID NO: 18 - DNA, 1098 bp, Artificial sequence
  cpo MDH2 from S. cerevisiae lacking first 12 a.a.
atgttgaagattgccatcttgggtgctgctggtggtatcggtcaatctttgtctttgttgttgaaggctcaattgcaataccaattgaaggaatccaacagatctgttacccacattcatttggctttgtacgatgtcaaccaagaagctatcaacggtgtcactgctgacttgtctcacatcgataccccaatctctgtttcctctcactctccagctggtggtattgaaaactgtttgcacaacgcttccattgttgtcattccagccggtgttccaagaaagccaggtatgacccgtgacgatttgttcaacgtcaatgccggtatcatctctcaattaggtgattccattgctgaatgttgtgacttgtccaaggttttcgtcttggttatctccaacccagtcaactctttggttcctgttatggtttccaacatcttgaagaaccacccacaatccagaaactctggtattgaaagaagaatcatgggtgtcaccaaattggacattgtcagagcttccactttcttgagagaaatcaacattgaatctggtttgactccaagagtcaactccatgccagatgttccagttatcggtggtcactctggtgaaactatcatcccattattctctcaatctaacttcttgtccagattgaatgaagatcaattgaaatacttgattcaccgtgtccaatacggtggtgacgaagttgtcaaggccaagaacggtaagggttctgctactctatccatggctcatgccggttacaagtgtgttgtccaattcgtttctctattattaggtaacattgaacaaatccacggtacctactacgttccattgaaagatgctaacaacttcccaattgctccaggtgctgaccaattattgccattagtcgacggtgctgactactttgccatcccattgaccatcactaccaagggtgtttcttacgttgactacgatatcgtcaacagaatgaacgacatggaaagaaaccaaatgttgcctatctgtgtttctcaattgaagaagaacattgacaagggtttggaattcgttgcttccagatctgcttccagttaa

SEQ ID NO: 19 - DNA, 2637 bp, Artificial sequence
  Synthetic construct TDH3p-delta12N MDH2-TDH3t for expression in S. cerevisiae
ggatccggcgcgccctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgttgaagattgccatcttgggtgctgctggtggtatcggtcaatctttgtctttgttgttgaaggctcaattgcaataccaattgaaggaatccaacagatctgttacccacattcatttggctttgtacgatgtcaaccaagaagctatcaacggtgtcactgctgacttgtctcacatcgataccccaatctctgtttcctctcactctccagctggtggtattgaaaactgtttgcacaacgcttccattgttgtcattccagccggtgttccaagaaagccaggtatgacccgtgacgatttgttcaacgtcaatgccggtatcatctctcaattaggtgattccattgctgaatgttgtgacttgtccaaggttttcgtcttggttatctccaacccagtcaactctttggttcctgttatggtttccaacatcttgaagaaccacccacaatccagaaactctggtattgaaagaagaatcatgggtgtcaccaaattggacattgtcagagcttccactttcttgagagaaatcaacattgaatctggtttgactccaagagtcaactccatgccagatgttccagttatcggtggtcactctggtgaaactatcatcccattattctctcaatctaacttcttgtccagattgaatgaagatcaattgaaatacttgattcaccgtgtccaatacggtggtgacgaagttgtcaaggccaagaacggtaagggttctgctactctatccatggctcatgccggttacaagtgtgttgtccaattcgtttctctattattaggtaacattgaacaaatccacggtacctactacgttccattgaaagatgctaacaacttcccaattgctccaggtgctgaccaattattgccattagtcgacggtgctgactactttgccatcccattgaccatcactaccaagggtgtttcttacgttgactacgatatcgtcaacagaatgaacgacatggaaagaaaccaaatgttgcctatctgtgtttctcaattgaagaagaacattgacaagggtttggaattcgttgcttccagatctgcttccagttaaggcccgggcgtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagatgcagtaatatacacagattccggccggccgcggccgc

SEQ ID NO: 20 - protein, 472 aa, Artificial sequence
  Amino acid sequence of fumarase from R. oryzae lacking first 23 a.a. + new methionine
MSSASAALQKFRAERDTFGDLQVPADRYWGAQTQRSLQNFDIGGPTERMPEPLIRAFGVLKKAAATVNMTYGLDPKVGEAIQKAADEVIDGSLIDHFPLVVWQTGSGTQTKMNVNEVISNRAIELLGGELGSKAPVHPNDHVNMSQSSNDTFPTAMHVAAVVEIHGRLIPALTTLRDALQAKSAEFEHIIKIGRTHLQDATPLTLGQEFSGYTQQLTYGIARVQGTLERLYNLAQGGTAVGTGLNTRKGFDAKVAEAIASITGLPFKTAPNKFEALAAHDALVEAHGALNTVACSLMKIANDIRYLGSGPRCGLGELSLPENEPGSSIMPGKVNPTQCEAMTMVCAQVMGNNTAISVAGSNGQFELNVFKPVMIKNLIQSIRLISDASISFTKNCVVGIEANEKKISSIMNESLMLVTALNPHIGYDKAAKCAKKAHKEGTTLKEAALSLGYLTSEEFDQWVRPEDMISAKD

SEQ ID NO: 21 - DNA, 1419 bp, Artificial sequence
  Nucleotide sequence of fumarase from R. oryzae encoding a protein lacking first 23 a.a.
atgtcctctgcttctgctgctttgcaaaaattcagagctgaaagagataccttcggtgacttgcaagttccagctgaccgttactggggtgctcaaactcaaagatctttgcaaaactttgacattggtggtccaactgaaagaatgccagaaccattaatcagagctttcggtgttttgaagaaggctgctgccaccgtcaacatgacctacggtttggacccaaaggttggtgaagccatccaaaaggctgctgacgaagttatcgatggttctttgattgaccatttcccattggttgtctggcaaaccggttctggtactcaaaccaagatgaacgtcaatgaagtcatctccaacagagccattgaattgttgggtggtgaattaggttccaaggctccagtccacccaaacgatcatgtcaacatgtctcaatcttccaacgacactttcccaactgccatgcacgttgctgccgttgttgaaattcacggtagattgattccagctttgaccactttgagagatgctttgcaagccaaatctgctgaattcgaacacatcatcaagattggtagaacccacttgcaagatgctaccccattgactttaggtcaagaattctccggttacactcaacaattgacctacggtattgctcgtgttcaaggtactttggaaagattatacaacttggctcaaggtggtactgctgtcggtactggtttgaacaccagaaagggtttcgatgccaaggttgctgaagccattgcttccatcactggtttaccattcaagaccgctccaaacaaattcgaagctttggctgctcacgacgctttggttgaagctcacggtgctttgaacaccgttgcttgttctttgatgaagattgccaacgatatccgttacttgggttctggtccaagatgtggtttaggtgaattgtctctaccagaaaacgaaccaggttcttccatcatgccaggtaaggtcaacccaactcaatgtgaagctatgaccatggtttgtgctcaagtcatgggtaacaacactgccatctctgttgctggttccaacggtcaattcgaattgaatgtctttaaaccagtcatgatcaagaacttgatccaatccatcagattaatctctgacgcttccatctctttcaccaagaactgtgttgtcggtattgaagctaacgaaaagaagatctcctccatcatgaacgaatctttgatgttggtcactgctttgaaccctcacattggttacgacaaggctgccaagtgtgccaagaaggctcacaaggaaggtaccactttgaaagaagctgctctatctttgggttacttgacctctgaagaattcgaccaatgggttagacctgaggacatgatttctgccaaggattaa

SEQ ID NO: 22 - DNA, 2950 bp, Artificial sequence
  Synthetic construct TDH1p-FUMR-TDH1t for expression in S. cerevisiae
ggatcccttcccttttacagtgcttcggaaaagcacagcgttgtccaagggaacaatttttcttcaagttaatgcataagaaatatctttttttatgtttagctaagtaaaagcagcttggagtaaaaaaaaaaatgagtaaatttctcgatggattagtttctcacaggtaacataacaaaaaccaagaaaagcccgcttctgaaaactacagttgacttgtatgctaaagggccagactaatgggaggagaaaaagaaacgaatgtatatgctcatttacactctatatcaccatatggaggataagttgggctgagcttctgatccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaaatgtcctctgcttctgctgctttgcaaaaattcagagctgaaagagataccttcggtgacttgcaagttccagctgaccgttactggggtgctcaaactcaaagatctttgcaaaactttgacattggtggtccaactgaaagaatgccagaaccattaatcagagctttcggtgttttgaagaaggctgctgccaccgtcaacatgacctacggtttggacccaaaggttggtgaagccatccaaaaggctgctgacgaagttatcgatggttctttgattgaccatttcccattggttgtctggcaaaccggttctggtactcaaaccaagatgaacgtcaatgaagtcatctccaacagagccattgaattgttgggtggtgaattaggttccaaggctccagtccacccaaacgatcatgtcaacatgtctcaatcttccaacgacactttcccaactgccatgcacgttgctgccgttgttgaaattcacggtagattgattccagctttgaccactttgagagatgctttgcaagccaaatctgctgaattcgaacacatcatcaagattggtagaacccacttgcaagatgctaccccattgactttaggtcaagaattctccggttacactcaacaattgacctacggtattgctcgtgttcaaggtactttggaaagattatacaacttggctcaaggtggtactgctgtcggtactggtttgaacaccagaaagggtttcgatgccaaggttgctgaagccattgcttccatcactggtttaccattcaagaccgctccaaacaaattcgaagctttggctgctcacgacgctttggttgaagctcacggtgctttgaacaccgttgcttgttctttgatgaagattgccaacgatatccgttacttgggttctggtccaagatgtggtttaggtgaattgtctctaccagaaaacgaaccaggttcttccatcatgccaggtaaggtcaacccaactcaatgtgaagctatgaccatggtttgtgctcaagtcatgggtaacaacactgccatctctgttgctggttccaacggtcaattcgaattgaatgtctttaaaccagtcatgatcaagaacttgatccaatccatcagattaatctctgacgcttccatctctttcaccaagaactgtgttgtcggtattgaagctaacgaaaagaagatctcctccatcatgaacgaatctttgatgttggtcactgctttgaaccctcacattggttacgacaaggctgccaagtgtgccaagaaggctcacaaggaaggtaccactttgaaagaagctgctctatctttgggttacttgacctctgaagaattcgaccaatgggttagacctgaggacatgatttctgccaaggattaaggcccgggcataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgcctttgcaaaacgcacatacccacaatatgtgactattggcaaagaacgcattatcctttgaagaggtggatactgatactaagagagtctctattccggctccacttttagtccagagattacttgtcttcttacgtatcagaacaagaaagcatttccaaagtaattgcatttgcccttgagcagtatatatatactaagaaggcgcgccgcggccgc

SEQ ID NO: 23 - protein, 1164 aa, Artificial sequence
  FRDm1 amino acid sequence from T. brucei lacking 68 a.a. N-terminal targeting sequence + new methionine
MADGISSASIVVTDPEAAAKKRDRMARELLSSNSGLCQEDEPTIINLKGLEHTIPYRLAVVLCNSRSTGEFEAKAAEILRKAFHMVDYSLNCFNPESELSRVNSLPVGEKHQMSEDLRHVMECTISVHHSSGMGFDPAAGPIISRLRGAMRDHNDMSDISVTEAEVELFSLAQSFDVDLEEGTIARKHSEARLDLGGVNKGYTVDYVVDHLRAAGMPNVLFEWGGDIRASGRNIKGNLWAVAIKRPPSVEEVIRRAKGKMLKMGEEEQEEKDDDSPSLLHVVELDDEALCTSGDYENVLYHPKHGVAGSIFDWQRRGLLSPEEGALAQVSVKCYSAMYADALATVCLVKRDAVRIRYLLEGWRYVRSRVTNYFAYTRQGERLAHMHEIAQETRELREIRIAGSLPSRIVIVGGGLAGLSAAIEAASCGAQVILMEKEGRIGGNSAKATSGINGWGTRTQAKSDILDGGKYFERDTFLSGVGGTTDPALVKVLSVKSGDAIGWLTSLGVPLSVLSQLGGHSFKRTHRAPDKTDGTPLPIGHTIMRTLEDHIRNNLSERVTIMTHVSVTELLHETDTTPDGASEVRVTGVRYRDLSDVDGQPSKLLADAVVLATGGFSNDREENSLLCKYAPHLASFPTTNGPWATGDGVKLATSVGAKLVDMDKVQLHPTGLIDPKDPANTTKILGPEALRGSGGILLNKQGKRFVNELDLRSVVSKAINTQGNEYPGSGGCYFAYCVLNEDATNLFCGGALGFYGKKLGLFQRAETVEELAKLIGCDEGELRDTLEKYETCSKAKVACPVTGKVVFPCVVGTRGPYNVAFVTPSIHYTMGGCLISPAAEVLQEYKGLNILENHRPIRCLFGAGEVTGGVHGGNRLGGNSLLECVVFGKIAGDRAATILQKREIALSKTSWTSVVVRESRSGEQFGTGSRVLRFNLPGALQRTGLNLGEFVAIRGEWDGQQLVGYFSPITLPEDLGTISLLVRADKGTLKEWICALRPGDSVEIKACGGLRIDQDPVKKCLLFRNRPITRFALVAAGTGVAPMLQVIRAALKKPYVDTLESIRLIYAAEEYDTLTYRSILQRFAEEFPDKFVCNFVLNNPPEGWTGGVGFVNKKSLQKVLQPPSSEPLIVVCGPPVMQRDVKNELLSMGYDKELVHTVDGESGTL

SEQ ID NO: 24 - DNA, 3498 bp, Artificial sequence
  FRDm1 nucleotide sequence from T. brucei encoding a protein lacking 68 a.a. N-terminal targeting sequence
atgggtgctgatggtatttcttctgcttccattgttgttactgacccagaagctgctgccaagaagcgtgacagaatggccagagaattgttgtcctccaactctggtctatgtcaagaagatgaaccaaccatcatcaacttaaagggtttggaacacaccattccatacagattggccgttgttttgtgtaactccagatccactggtgaattcgaagccaaggctgctgaaatcttgagaaaggctttccacatggttgactactctttgaattgtttcaacccagaatctgaattgtcccgtgtcaactctttaccagtcggtgaaaagcaccaaatgtccgaagatctaagacatgtcatggaatgtaccatttctgtccaccactcctctggtatgggtttcgacccagctgctggtccaatcatctccagattgagaggtgccatgagagatcacaacgacatgtccgatatctccgtcactgaagctgaagttgaattattctctttggctcaatctttcgatgtcgacttggaagaaggtactattgccagaaagcactctgaagccagattggatttgggtggtgtcaacaagggttacactgttgactacgttgttgaccatttgagagctgctggtatgccaaacgtcttgttcgaatggggtggtgatatcagagcttctggtagaaacatcaagggtaacttgtgggctgttgccatcaagcgtccaccatctgttgaagaagttatccgtcgtgccaagggtaagatgttaaagatgggtgaagaagaacaagaagaaaaggacgatgactctccatctttgttgcacgttgttgaattggatgacgaagctttgtgtacctctggtgactacgaaaacgtcttataccatccaaagcacggtgttgctggttccattttcgactggcaacgtcgtggtttattgtctccagaagaaggtgctttagctcaagtttccgtcaaatgttactctgccatgtacgctgatgctttggccactgtttgtttggtcaagagagatgctgtcagaatcagatacttgttggaaggttggagatacgtcagatctcgtgtcaccaactacttcgcttacaccagacaaggtgaaagattggctcacatgcacgaaattgctcaagaaaccagagaattaagagaaatcagaattgctggttctttgccatccagaattgttatcgtcggtggtggtttggctggtctatccgctgccattgaagctgcttcttgtggtgctcaagtcattttgatggaaaaggaaggtagaattggtggtaactctgccaaggctacctctggtatcaacggttggggtaccagaacccaagccaagtctgatatcttggatggtggtaagtactttgaaagagacactttcttgtccggtgtcggtggtaccactgacccagctttggtcaaggtcttgtccgtcaaatctggtgacgctatcggttggttaacttctttgggtgtcccattgtccgttttgtctcaattgggtggtcactctttcaagagaactcacagagctccagacaagactgatggtactccattaccaattggtcacaccatcatgagaactttggaagatcatatcagaaacaacttgtctgaaagagttaccatcatgacccacgtttctgttactgaattgttgcacgaaactgacaccactccagatggtgcttctgaagttcgtgtcaccggtgtccgttacagagacttgtctgatgtcgatggtcaaccttccaaactattggctgacgctgttgttttggccactggtggtttctccaacgacagagaagaaaactctttgttgtgtaaatacgctcctcatttggcttctttcccaactaccaacggtccatgggctactggtgacggtgtcaaattggccacctccgttggtgccaagttggttgacatggacaaggttcaattgcacccaactggtttgattgacccaaaggacccagctaacaccactaagatcttgggtccagaagctttgagaggttctggtggtattttgttgaacaagcaaggtaagagattcgtcaacgaattggacttgagatccgttgtttccaaggccattaacactcaaggtaacgaatacccaggttctggtggttgttactttgcttactgtgtcttaaacgaagatgctaccaacttattctgtggtggtgctttgggtttctacggtaagaaattaggtttgttccaaagagctgaaactgttgaagaattggccaaattgattggttgtgacgaaggtgaattgagagacactttggaaaaatacgaaacctgttccaaggccaaggttgcttgtccagtcactggtaaggttgttttcccatgtgttgtcggtaccagaggtccatacaatgttgctttcgtcactccatccatccactacaccatgggtggttgtttgatctctccagctgctgaagtcttgcaagaatacaagggtttgaatatcttggaaaaccacagaccaatcagatgtttgttcggtgctggtgaagtcactggtggtgtccacggtggtaacagattaggtggtaactctctattggaatgtgttgtctttggtaagattgctggtgacagagctgccactatcttgcaaaagagagaaattgctttgtccaagacctcctggacctctgttgttgtcagagaatccagatctggtgaacaattcggtaccggttccagagttttgagattcaacttgccaggtgctttacaaagaaccggtttgaacttgggtgaattcgttgccatcagaggtgaatgggatggtcaacaattagtcggttacttctctccaatcactttgccagaagatttgggtaccatctctttgttggtcagagctgacaagggtactttgaaggaatggatctgtgctttgcgtccaggtgactccgttgaaatcaaggcttgtggtggtctaagaattgaccaagatccagtcaagaaatgtttgttgttcagaaacagaccaattaccagatttgctttggttgctgctggtaccggtgttgctccaatgttgcaagttatcagagctgctttgaagaagccatacgtcgacactttggaatccatcagattgatctacgctgctgaagaatatgacactttaacctacagatctatcttgcaaagatttgctgaagaattcccagacaaattcgtttgtaacttcgtcttaaacaaccctccagaaggttggaccggtggtgttggtttcgtcaacaagaaatctttgcaaaaggttttgcaaccaccttcttctgaaccattgattgttgtttgtggtccacctgttatgcaaagagatgtcaaaaatgaattgttgtccatgggttacgacaaggaattggttcacactgtcgatggtgaatctggtaccttgtaa

SEQ ID NO: 25 - DNA, 5037 bp, Artificial sequence
  Synthetic construct TDH3p-FRDm1-TDH3t for expression in S. cerevisiae
ggatccggcgcgccctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgggtgctgatggtatttcttctgcttccattgttgttactgacccagaagctgctgccaagaagcgtgacagaatggccagagaattgttgtcctccaactctggtctatgtcaagaagatgaaccaaccatcatcaacttaaagggtttggaacacaccattccatacagattggccgttgttttgtgtaactccagatccactggtgaattcgaagccaaggctgctgaaatcttgagaaaggctttccacatggttgactactctttgaattgtttcaacccagaatctgaattgtcccgtgtcaactctttaccagtcggtgaaaagcaccaaatgtccgaagatctaagacatgtcatggaatgtaccatttctgtccaccactcctctggtatgggtttcgacccagctgctggtccaatcatctccagattgagaggtgccatgagagatcacaacgacatgtccgatatctccgtcactgaagctgaagttgaattattctctttggctcaatctttcgatgtcgacttggaagaaggtactattgccagaaagcactctgaagccagattggatttgggtggtgtcaacaagggttacactgttgactacgttgttgaccatttgagagctgctggtatgccaaacgtcttgttcgaatggggtggtgatatcagagcttctggtagaaacatcaagggtaacttgtgggctgttgccatcaagcgtccaccatctgttgaagaagttatccgtcgtgccaagggtaagatgttaaagatgggtgaagaagaacaagaagaaaaggacgatgactctccatctttgttgcacgttgttgaattggatgacgaagctttgtgtacctctggtgactacgaaaacgtcttataccatccaaagcacggtgttgctggttccattttcgactggcaacgtcgtggtttattgtctccagaagaaggtgctttagctcaagtttccgtcaaatgttactctgccatgtacgctgatgctttggccactgtttgtttggtcaagagagatgctgtcagaatcagatacttgttggaaggttggagatacgtcagatctcgtgtcaccaactacttcgcttacaccagacaaggtgaaagattggctcacatgcacgaaattgctcaagaaaccagagaattaagagaaatcagaattgctggttctttgccatccagaattgttatcgtcggtggtggtttggctggtctatccgctgccattgaagctgcttcttgtggtgctcaagtcattttgatggaaaaggaaggtagaattggtggtaactctgccaaggctacctctggtatcaacggttggggtaccagaacccaagccaagtctgatatcttggatggtggtaagtactttgaaagagacactttcttgtccggtgtcggtggtaccactgacccagctttggtcaaggtcttgtccgtcaaatctggtgacgctatcggttggttaacttctttgggtgtcccattgtccgttttgtctcaattgggtggtcactctttcaagagaactcacagagctccagacaagactgatggtactccattaccaattggtcacaccatcatgagaactttggaagatcatatcagaaacaacttgtctgaaagagttaccatcatgacccacgtttctgttactgaattgttgcacgaaactgacaccactccagatggtgcttctgaagttcgtgtcaccggtgtccgttacagagacttgtctgatgtcgatggtcaaccttccaaactattggctgacgctgttgttttggccactggtggtttctccaacgacagagaagaaaactctttgttgtgtaaatacgctcctcatttggcttctttcccaactaccaacggtccatgggctactggtgacggtgtcaaattggccacctccgttggtgccaagttggttgacatggacaaggttcaattgcacccaactggtttgattgacccaaaggacccagctaacaccactaagatcttgggtccagaagctttgagaggttctggtggtattttgttgaacaagcaaggtaagagattcgtcaacgaattggacttgagatccgttgtttccaaggccattaacactcaaggtaacgaatacccaggttctggtggttgttactttgcttactgtgtcttaaacgaagatgctaccaacttattctgtggtggtgctttgggtttctacggtaagaaattaggtttgttccaaagagctgaaactgttgaagaattggccaaattgattggttgtgacgaaggtgaattgagagacactttggaaaaatacgaaacctgttccaaggccaaggttgcttgtccagtcactggtaaggttgttttcccatgtgttgtcggtaccagaggtccatacaatgttgctttcgtcactccatccatccactacaccatgggtggttgtttgatctctccagctgctgaagtcttgcaagaatacaagggtttgaatatcttggaaaaccacagaccaatcagatgtttgttcggtgctggtgaagtcactggtggtgtccacggtggtaacagattaggtggtaactctctattggaatgtgttgtctttggtaagattgctggtgacagagctgccactatcttgcaaaagagagaaattgctttgtccaagacctcctggacctctgttgttgtcagagaatccagatctggtgaacaattcggtaccggttccagagttttgagattcaacttgccaggtgctttacaaagaaccggtttgaacttgggtgaattcgttgccatcagaggtgaatgggatggtcaacaattagtcggttacttctctccaatcactttgccagaagatttgggtaccatctctttgttggtcagagctgacaagggtactttgaaggaatggatctgtgctttgcgtccaggtgactccgttgaaatcaaggcttgtggtggtctaagaattgaccaagatccagtcaagaaatgtttgttgttcagaaacagaccaattaccagatttgctttggttgctgctggtaccggtgttgctccaatgttgcaagttatcagagctgctttgaagaagccatacgtcgacactttggaatccatcagattgatctacgctgctgaagaatatgacactttaacctacagatctatcttgcaaagatttgctgaagaattcccagacaaattcgtttgtaacttcgtcttaaacaaccctccagaaggttggaccggtggtgttggtttcgtcaacaagaaatctttgcaaaaggttttgcaaccaccttcttctgaaccattgattgttgtttgtggtccacctgttatgcaaagagatgtcaaaaatgaattgttgtccatgggttacgacaaggaattggttcacactgtcgatggtgaatctggtaccttgtaaggcccgggcgtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagatgcagtaatatacacagattccggccggccgcggccgc